(19) United States
(12) Patent Application Publication
(10) Pub. No.: US 2014/0074106 A1
(43) Pub. Date: Mar. 13, 2014

(54) WORKING TOWER, ROD INSERTER, ROD REDUCER, AND COMPRESSION-DISTRACTION TOOL FOR MINIMALLY INVASIVE SURGERY SYSTEM

(71) Applicant: GS MEDICAL CO., LTD., Chungcheongbuk-do (KR)

(72) Inventor: Min-Sik Shin, Seoul (KR)

(73) Assignee: GS MEDICAL CO., LTD., Chungcheongbuk-do (KR)

(21) Appl. No.: 13/653,400

(22) Filed: Oct. 16, 2012

(30) Foreign Application Priority Data
Sep. 12, 2012 (KR) 10-2012-0100966

(51) Int. Cl. A61B 17/70

(52) U.S. Cl. 606/104

(57) ABSTRACT

A working tower for gripping a correction screw during minimally invasive surgery and playing a role of a working surface for other external devices is provided, which includes an exterior pipe, an interior pipe slidably provided within the exterior pipe, a driving sleeve rotatably connected to the exterior pipe and screw-coupled to the interior pipe to move the interior pipe upward and downward along a lengthwise direction thereof during rotation, and a screw holder provided on a lower end of the interior pipe, and is elastically split apart to receive a head portion of the correction screw when withdrawn from the exterior pipe, and contracted to securely hold the head portion when inserted into the exterior pipe.

### CROSS-REFERENCE TO RELATED APPLICATIONS

This application claims priority from Korean Patent Application No. 2012-0100966, filed on Sep. 12, 2012, in the Korean Intellectual Property Office, the disclosure of which is incorporated herein by reference in its entirety.

### BACKGROUND

1. Field of the Invention

The invention relates to a working tower, a rod inserter, a rod reducer, and a compression-distraction tool for a minimally invasive surgery system.

2. Description of the Related Art

Generally, vertebral column (spine) consists of 24 vertebrae (excluding sacrovertebra and caudal vertebra), and plays a role of supporting posture of a body and body movements, and importantly, protecting internal organs.

There is intervertebral disc between vertebrae which works as a buffer to absorb shock exerted thereto. The spinal cord runs through the vertebrae of the spine.

Due to the structure of the spine, disc between vertebrae is damaged when exposed to incorrect posture for a prolonged time, or due to age-related degenerative diseases or external shock, and intervertebral disc disease may develop. The intervertebral disc disease is generally accompanied with acute pain due to pressed spinal chord.

The following procedure is generally performed to treat the intervertebral disc disease.

A practitioner removes disc from affected site so that the site is freed from pressure or compression, inserts an artificial substitute replacement (e.g., cage) filled with bone chips, and corrects the position of the vertebrae above and below the damaged disc. Furthermore, in order to correct the position of the vertebrae, a surgeon couples screws on the vertebrae above and below the damaged disc, connects the coupled screws with rods, corrects the position of the vertebrae, and couples the rod and the screws with a set screw. As a result, fusion normally occurs between the vertebrae in the straightened position.

However, considering the downside of the above-mentioned surgery, that is, slow recovery due to relatively large invasive area, and low satisfaction due to wounds after surgery, minimally invasive surgery has been developed.

Korean Patent No. 10-0942226 registered to the present Applicant is drawn to a technology related to the minimally invasive surgery, which teaches a rod holder and a vertebral minimally invasive surgical system using the same.

Referring to FIGS. 1A and 1B, the conventional system of KR10-0942226 includes rod guides 60 to be inserted into a patient's skin, which is punctured by a separate puncturing tool (not illustrated), and to guide the rods 20 and also grips pedicle screws 10, a rod holder 30 loaded with the rods 20 to insert the same to cutaway portions 61a of the rod guides 60, a rod pusher 70 to press the rods 20 downward as much as possible so that the rods 20 are brought into contact with bottoms of receiving grooves 11 of the pedicle screws 10, and a space adjusting portion 95 (FIG. 1B) to adjust the distance between the rod guides 60.

However, since the conventional rod guide 60 has a structure in which the gripping portion 61b of an interior body 61 is not allowed to be completely inserted into an external sleeve 65 due to the presence of a rod pressing portion 5a protruding from a distal end of the external sleeve 65, it is necessary to minimize the opening portion of the gripping portions 61b before insertion to compensate for the decrease of gripping force due to incompletely inserted portion. That is, the practitioner has to force-fit the pedicle screw 10 into the gripping portion 61b which can be cumbersome. Further, structure is complex due to a need for a separate rod guide separator (not illustrated) to efficiently separate the force-fit pedicle screw 10 from the gripping part 61b of the rod guide 60 after surgery.

Furthermore, because the conventional rod holder 30 is designed to be loaded with rod by two steps, it has complicated structure. Use of the conventional rod holder 30 is also inconvenient because it is necessary to separately manipulate a button portion 37 in addition to the manipulation to lift up or lower down the loading portion 35.

Furthermore, because the conventional rod pusher 70 does not have a function of coupling a set screw (not illustrated) to the pedicle screw 10, that is, because the conventional rod pusher 70 has a structure in which pushing down the rod 20 cannot be performed concurrently with the set screw coupling, there is a risk of having tissue caught between the rod 20 and the set screw during later process of coupling set screw. Further, the conventional rod pusher 70 is not a cannulated type which can be inserted into a hollow portion of the rod guide 60, but is placed outside the rod guide 60 which can be interfered with the other components.

Further, because the conventional space adjusting portion 95 (FIG. 1B) has a relatively slender cylindrical rod 95d for a pivot, the distance between the pedicle screws 10 that can be adjusted by the space adjusting portion 95 is limited. Further, there is possibility that the rod guide 60 can move in the lengthwise direction of the cylindrical rod 95d when gripped. Regarding the components of the drawings that are not mentioned, a gripping portion 95a grips the rod guide 60, and handles 95b, 95c tighten the gripping portion.

### SUMMARY

Exemplary embodiments of the present inventive concept overcome the above disadvantages and other disadvantages not described above. Also, the present inventive concept is not required to overcome the disadvantages described above, and an exemplary embodiment of the present inventive concept may not overcome any of the problems described above.

According to one embodiment, a technical objective is to provide a working tower as an improvement over a conventional rod guide, which provides convenience of use since a correction screw is easily mountable thereon and demounted without requiring use of a separate separating device.

Another technical object is to provide a rod inserter as an improvement over a conventional rod holder, which has simpler structure and easier to manipulate.

Another technical object is to provide a rod reducer as an improvement over a conventional rod pusher, according to which accident of tissue being caught between rod and set screw can be prevented because it is possible to couple the set crew to the correction screw while pressing on the rod, and according to which it is also possible to minimize interference with the other components because the rod reducer is designed to be inserted into another component.

Yet another technical object is to provide a compression-distraction tool as an improvement over a conventional space adjusting portion, which provides a wide range of space displacement adjustment for compression and distraction between the correction screws and which keeps a component located on a pivot from moving in a lengthwise direction of the pivot.

In one embodiment, a working tower for gripping a correction screw during minimally invasive surgery and playing a role of a working surface for other external devices, is provided, which may include an exterior pipe, an interior pipe slidably provided within the exterior pipe, a driving sleeve rotatably connected to the exterior pipe and screw-coupled to the interior pipe to move the interior pipe upward and downward along a lengthwise direction thereof during rotation, and a screw holder provided on a lower end of the interior pipe, and is elastically split apart to receive a head portion of the correction screw when withdrawn from the exterior pipe, and contracted to securely hold the head portion when inserted into the exterior pipe.

In one embodiment, a rod inserter for gripping a rod and seating the rod on a rod accommodating part of at least two correction screws during a minimally invasive surgery, is provided, which may include a fixed bar, a moving bar slidably provided on the fixed bar, an adjusting knob rotatably connected to the fixed bar and screw-coupled to the moving bar to adjust a moving distance of the moving bar by rotation thereof, and a rod holder provided between one end of the fixed bar and one end of the moving bar to grip the rod in accordance with a movement of the moving bar.

In one embodiment, a rod reducer for pressing down a rod seated in a rod accommodating part of a correction screw and concurrently coupling a set screw to the rod accommodating part during a minimally invasive surgery, is provided, which may include an exterior member in a tubular form comprising a set screw mounting part formed on a lower end thereof, an interior member slidably provided inside the exterior member, the interior member comprising a lower withdrawing part formed on a lower end thereof which is in a state of being withdrawn from the exterior member into a contact with the rod, and a compressing sleeve for coupling for descending the interior member while being coupled to a third external device gripping the correction screw so that the rod is compressed by the lower withdrawing part of the interior member, and a rotation handle for rotating the exterior member so that the set screw is screw-coupled to the rod accommodating part of the correction screw.

In one embodiment, a compression-distraction tool for spreading a first and a second correction screws on lower ends of a first and a second working towers from each other in a distraction mode, or approaching the first and second correction screws to each other in a compression mode in a minimally invasive surgery, is provided, which may include a pivot positioned between the first and second working towers, a tower compressing unit provided on one end of the pivot to compress the first and second working towers so that the first and second working towers are tilt with reference to the pivot, and a tower supporter rotatably provided on the other end of the pivot to prevent movement of the first and second working towers in a lengthwise direction of the pivot.

According to the first embodiment of the invention, it is easy to use the working tower because, compared to the conventional rod guide, a correction screw is easily mountable and removable without requiring a use of a separate separator.

According to the second embodiment of the invention, a rod inserter with simpler structure and easier operation than the conventional rod holder is provided.

According to the third embodiment of the invention, compared to the conventional rod pusher, the rod reducer can prevent a possibility that tissues are caught in between the rod and the set screw thanks to a structure which enables to press the rod and at the same time coupled the set screw to the correction screw, and can also minimize interference with the adjacent members thanks to a structure thereof which is insertable into another member.

According to the fourth embodiment of the invention, compared to the conventional space adjusting tool, the compression-distraction tool can provide a wide range of displacement adjustment for distraction and compression between the correction screws, and can also prevent the possibility that a member positioned on a pivot is moved in a lengthwise direction of the pivot.

### BRIEF DESCRIPTION OF THE DRAWINGS

The above and/or other aspects of the present inventive concept will be more apparent by describing certain exemplary embodiments of the present inventive concept with reference to the accompanying drawings, in which:

FIGS. 1A and 1B are perspective views of components of a conventional minimally invasive surgical system;

FIG. 2 is a perspective view of a working tower according to a first embodiment of the invention;

FIG. 3 is a cross-section view illustrating the working tower of FIG. 2 which has branched-apart portions;

FIG. 4 is a longitudinal cross-section illustrating the gripping portion inserted in the state of FIG. 3;

FIG. 5 is a cross-section view illustrating interior of an exterior pipe of the working tower of FIG. 2;

FIG. 6 is a cross section view taken on line VI-VI of the working tower of FIG. 3;

FIG. 7 is a perspective view of a rod inserter according to a second embodiment of the invention;

FIG. 8 is a front view of the rod inserter of FIG. 7;

FIG. 9 is a cross section view taken on line IX-IX of the rod inserter of FIG. 8;

FIG. 10 is a plan view of encircled portion “A” of FIG. 8;

FIG. 11 is a cross-section view taken on line XI-XI of the rod inserter of FIG. 8;

FIG. 12 is a cross-section view taken on line XII-XII of the rod inserter of FIG. 8;

FIGS. 13A and 13B are views illustrating the states before and after the rod inserter of FIG. 7 grips a center of the rod;

FIG. 14 is a schematic perspective view illustrating the rod inserter of FIG. 7, gripping the center of the rod, inserting a rod into a rod guiding groove of the working tower;

FIGS. 15A and 15B are views illustrating the states before and after the rod inserter of FIG. 7 grips an end of the rod;

FIG. 16 is a schematic perspective view illustrating the rod inserter of FIG. 7, gripping the end of the rod, inserting a rod into a rod guiding groove of the working tower;

FIG. 17 is a front view illustrating a rod inserter according to a second embodiment of the invention;

FIG. 18 is a perspective view illustrating a rod holder of the rod inserter of FIG. 17;

FIG. 19 is a perspective view illustrating a cover being mounted on the rod inserter of FIG. 17;

FIG. 20 is a front view of the rod inserter of FIG. 19;

FIG. 21 is a cutaway view illustrating a main portion of the rod inserter of FIG. 20;

FIG. 22 is a perspective view illustrating a rod being mounted on the rod inserter of FIG. 19;

FIG. 23 is a perspective view illustrating a rod after mounted on the rod inserter of FIG. 19;

FIG. 24 is a perspective view illustrating the cover of the rod inserter moving from the state of FIG. 23 toward the clip, causing the clip locked;

FIG. 25 is a perspective view illustrating a rod being rotated as a moving bar is moved toward the clip in accordance with rotation of an adjusting knob in the state of FIG. 24;

FIG. 26 is a perspective view of a rod reducer according to a third embodiment of the invention;

FIG. 27 is a longitudinal cross-section view of the rod reducer of FIG. 26;

FIG. 28 is a schematic cross-section view illustrating a rod being pressed by a lower withdrawing part of an interior member while a coupling sleeve is rotated;

FIG. 29 is a schematic cross-section view illustrating a set screw being coupled into a rod inserting portion of a correction screw while a rotation handle is pressed and rotated;

FIG. 30 is a perspective view of a compression-distraction tool according to a fourth embodiment of the invention;

FIG. 31 is a plan view of the compression-distraction tool of FIG. 30;

FIG. 32 is a front view of the compression-distraction tool of FIG. 30;

FIG. 33 is a cross-section view taken on line XXXIII-XXXIII of the compression-distraction tool of FIG. 31;

FIG. 34 is a detailed perspective view of a screw rod or the like of the compression-distraction tool of FIG. 32;

FIG. 35 is a perspective view of the compression-distraction tool of FIG. 30 viewed from a front upper direction; and

FIGS. 36A and 36B are schematic views respectively illustrating the compression-distraction tool in use, i.e., in distraction and compression modes, respectively.

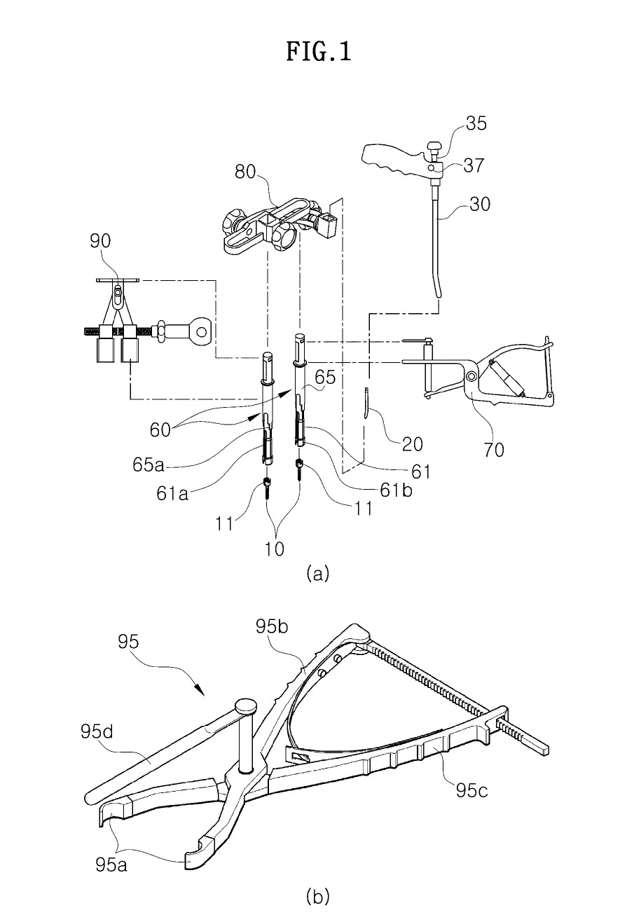

### DETAILED DESCRIPTION OF EXEMPLARY EMBODIMENTS

Certain exemplary embodiments of the present inventive concept will now be described in greater detail with reference to the accompanying drawings.

In the following description, same drawing reference numerals are used for the same elements even in different drawings. The matters defined in the description, such as detailed construction and elements, are provided to assist in a comprehensive understanding of the present inventive concept. Accordingly, it is apparent that the exemplary embodiments of the present inventive concept can be carried out without those specifically defined matters. Also, well-known functions or constructions are not described in detail since they would obscure the invention with unnecessary detail.

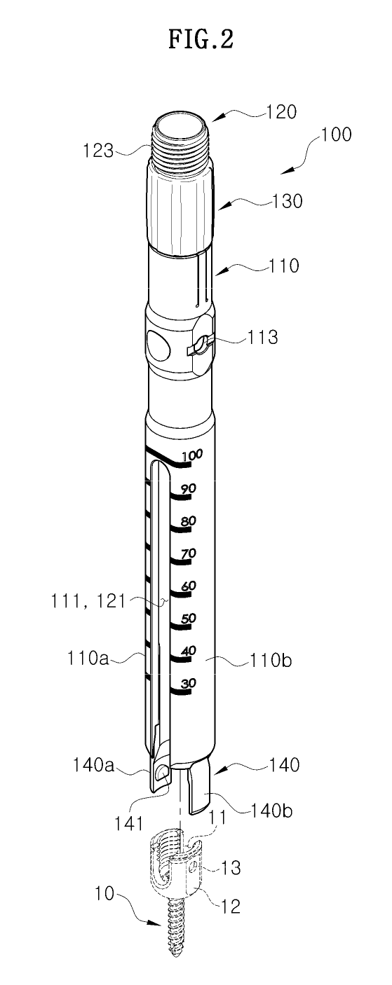

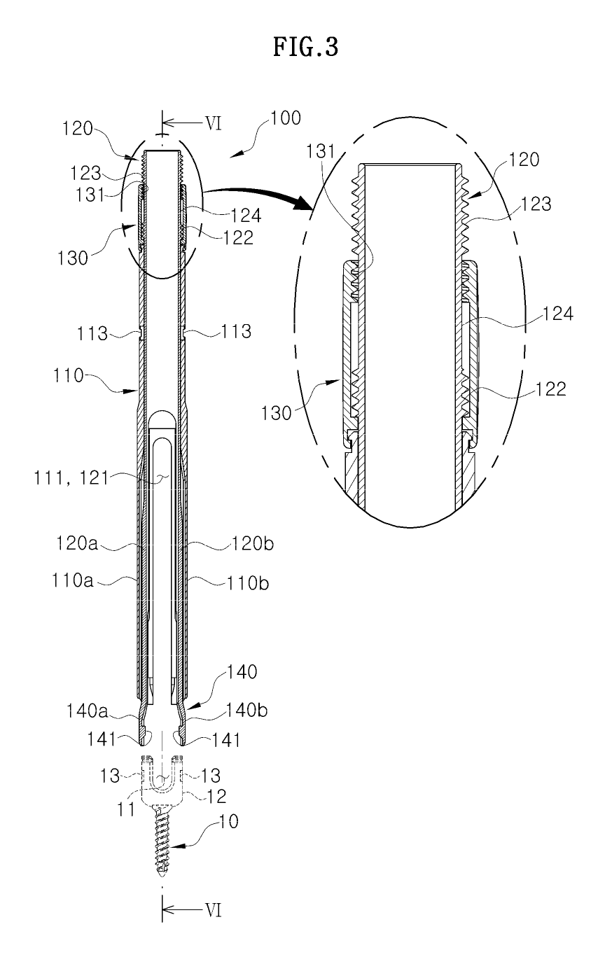

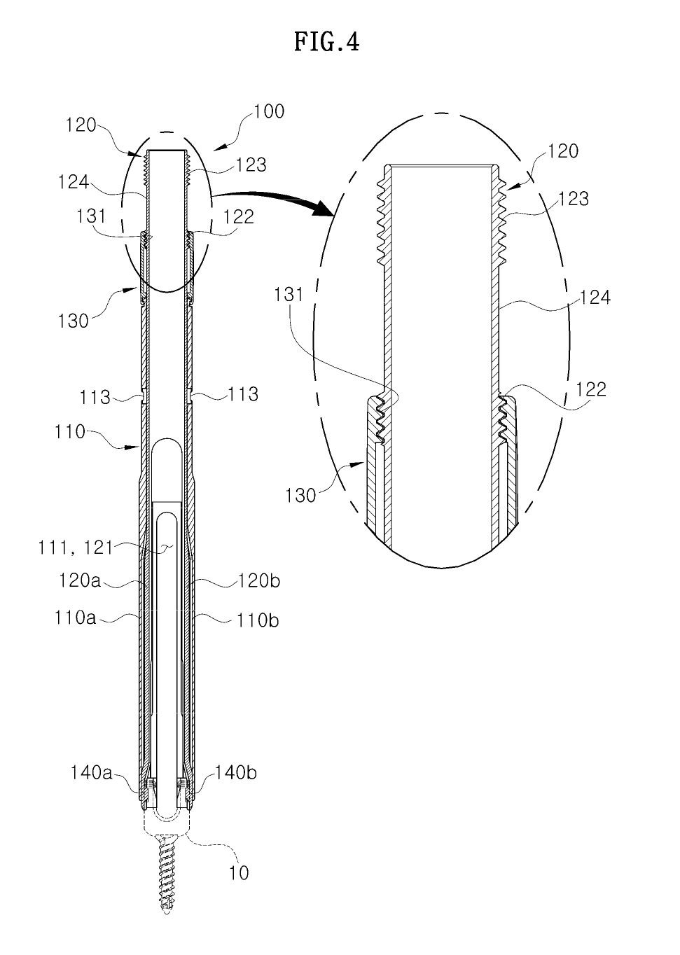

FIG. 2 is a perspective view of a working tower according to a first embodiment of the invention, FIG. 3 is a cross-section view illustrating the working tower of FIG. 2 which has branched-apart portions, and FIG. 4 is a longitudinal cross-section illustrating the gripping portion inserted in the state of FIG. 3.

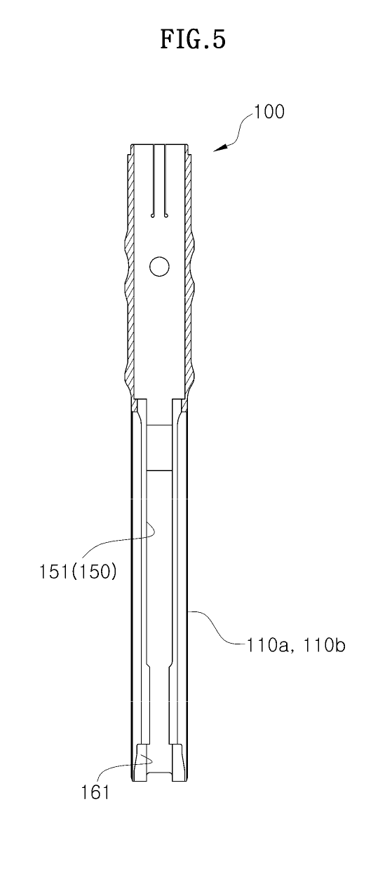

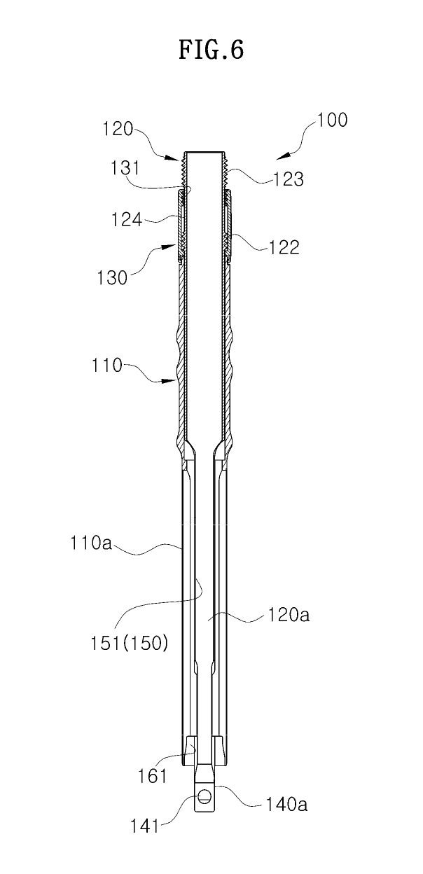

FIG. 5 is a cross-section view illustrating interior of an exterior pipe of the working tower of FIG. 2, and FIG. 6 is a cross section view taken on line VI-VI of the working tower of FIG. 3.

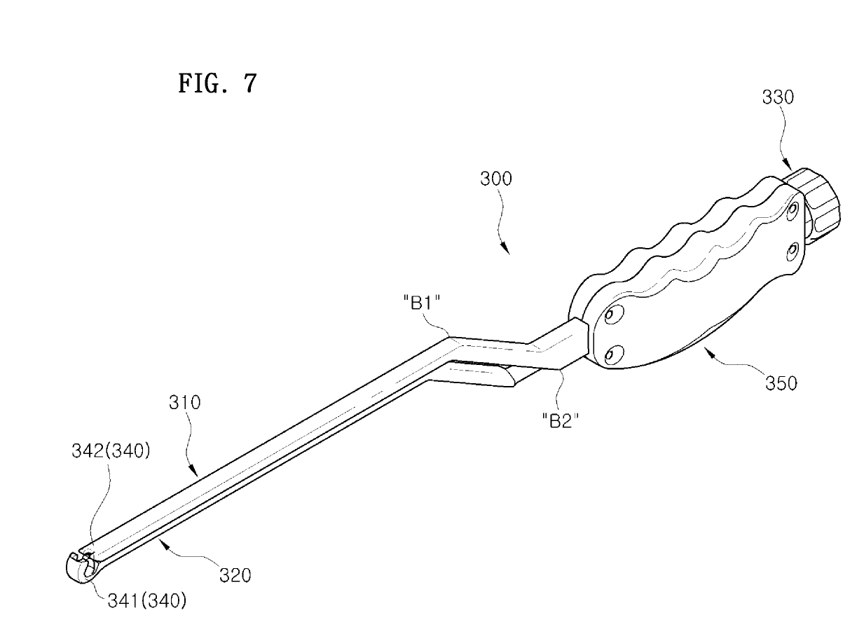

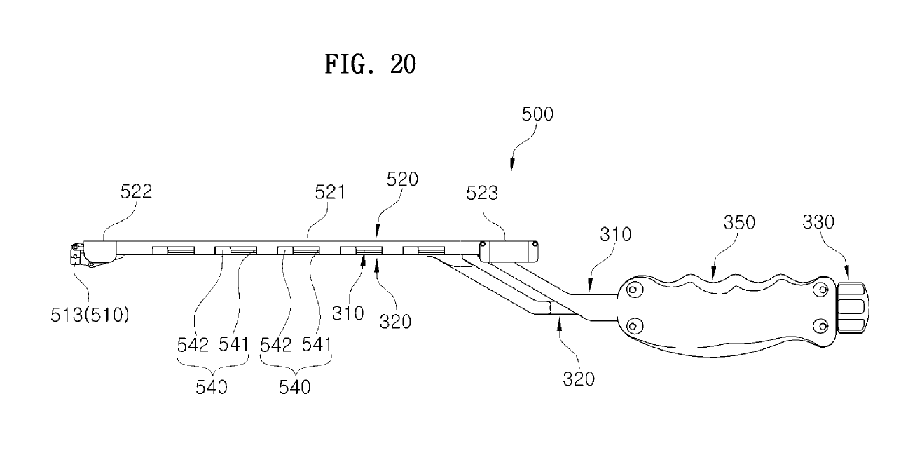

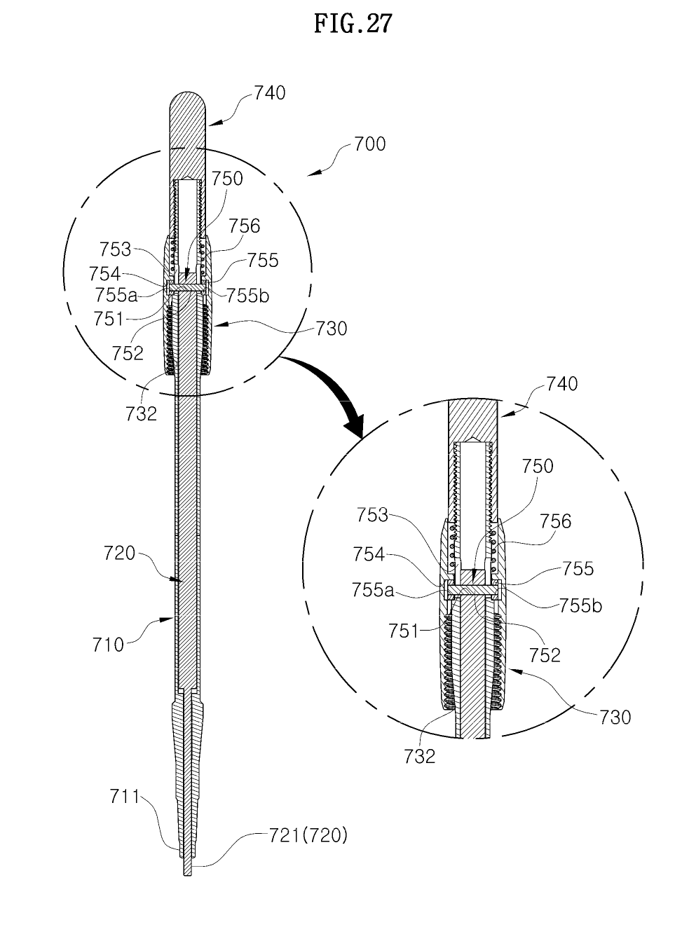

Referring to FIGS. 2 to 4, according to a first embodiment of the invention, the working tower 100 grips a correction screw 10, and plays a role of a working surface of the other external devices such as, for example, rod inserter 300, 500 (FIGS. 7, 20), rod reducer 700 (FIG. 27), or compression-distraction tool 900 (FIG. 31). The working tower 100 includes an exterior pipe 110, an interior pipe 120, a driving sleeve 130, and a screw holder 140.

The exterior pipe 110 is a tubular member forming an exterior side of the working tower 100. To be specific, a lower half of the exterior pipe 110 is branched into a first and a second exterior pipe branch portions 110a, 110b, and a first guide groove 111 may be formed between the first and second exterior pipe branch portions 110a, 110b.

The interior pipe 120 is slidably provided inside the exterior pipe 110 to be slid along a lengthwise direction thereof. To be specific, a lower half of the interior pipe 120 is branched into a first and a second interior pipe branch portions 120a, 120b, and a second guiding groove 121 corresponding to the first rod guide groove 111 may be formed between the first and second interior pipe branch portions 120a, 120b. Accordingly, the rod 20 can be efficiently moved to the rod receiving portion 11 of the correction screw 10 by use of the first and second rod guiding holes 111, 121. For reference, the rod 20 may be inserted into the first and second rod guiding grooves 111, 121 in a state of being gripped at the rod inserter 300 (FIG. 7).

Further, for screw coupling with the first external device (e.g., rod reducer 700 (FIG. 27)), a female screw 123 (‘first female screw’) for coupling with external device may be formed on an outer circumference of the interior pipe 120. By way of example, the inner circumference of the coupling sleeve 730 (FIG. 27) of the rod reducer 700 (FIG. 27) and the outer circumference of the interior pipe 120 may be screw-coupled with each other through the first female screw 123. Further, referring to FIG. 2, because the first female screw is formed above the female screw 122 for sleeve coupling (to be explained below), the rod reducer 700 (FIG. 27) is efficiently coupled with the first female screw 123 without being interfered with the female screw 122 for sleeve coupling during the coupling of the coupling sleeve 730 (FIG. 27) of the rod reducer 700 (FIG. 27).

Referring to FIG. 2, the driving sleeve 130 is rotated to move the interior pipe 120 upward and downward along a lengthwise direction thereof. The driving sleeve 130 is rotatably connected to the exterior pipe 110 and screw-coupled with the interior pipe. For screw-coupling with the interior pipe 120, mail screw 131 may be formed on an upper inner circumference of the driving sleeve 130, and a female screw 122 (‘second female screw’) for sleeve coupling may also be formed on an outer circumference of the interior pipe 120 to correspond to the male screw 131. Accordingly, when the driving sleeve 130 is rotated forward or backward in a state that the male screw 131 and the second female screw 122 are screw-coupled with each other, the interior pipe 120 is moved upward or downward.

Further, the second female screw 122 may be provided at a predetermined distance away from the first female screw 123. That is, a threadless section 124 may be formed between the second female screw 122 and the first female screw 123. By forming the threadless section 124, it is possible to insert or withdraw the screw holder 140 by pulling or pushing the interior pipe 120 without having to rotate the driving sleeve 130. As a result, time to rotate the driving sleeve 130 is saved because the rotating is replaced with pulling or pushing, and time for surgery is reduced. The ways to pull or push the interior pipe 120 will be explained in greater detail below.

The screw holder 140 is provided at a lower end of the interior pipe 120, and is elastically split apart to receive the head portion 120 of the correction screw 10 when withdrawn from the exterior pipe 110, and contracted to securely hold the head portion 12 therein when inserted into the exterior pipe 110. By way of example, the screw holder 140 may include a first and a second holding arms 140a, 140b provided on ends of the first and second interior pipe branch portions 120a, 120b. The first and second holding arms 140a, 140b may include a coupling portion 141 to be coupled with the head portion 12 of the correction screw 10. By way of example, if the head portion 12 of the correction screw 10 has a coupling recess 13 formed therein, the coupling portion 141 may be provided as a coupling protrusion. Alternatively, and although not illustrated herein, if the head portion 12 of the correction screw 10 has a coupling protrusion (not illustrated) formed thereon, corresponding coupling recesses (not illustrated) may be formed in the first and second holding arms 140a, 140b.

Further, if the first and second holding arms 140a, 140b are elastically split apart from each other as these are withdrawn from the exterior pipe 110, the distance between the first and second holding arms 140a, 140b may be set to be larger than an external diameter of the head portion 12 of the correction screw 10. Accordingly, when the working tower 100 according to the first embodiment of the invention is used for the minimally invasive spinal surgery, time for surgery is shortened, because the correction screw 10 is easily inserted into the first and second holding arms 140a, 140b without requiring the surgeon to separately force-fit the same.

Furthermore, referring to FIGS. 2 and 3, an external device inserting recess 113 may be formed in the outer circumference of the exterior pipe 110 to receive a second external device (e.g., fixing protrusion 922 (FIG. 31) of tower pressing unit 920 (FIG. 31) of the compression-distraction tool 700 (FIG. 31) to be explained below).

Furthermore, referring to FIGS. 5 and 6, the interior and exterior pipes 120, 110 may be slid upward or downward relative to each other without being rotated to the left or right along a pipe guiding portion 150. For example, the pipe guiding portion 150 may be provided as interior pipe guiding grooves 151 formed in the interior surfaces of the first and second exterior pipe branch portions 110a, 110b to guide the first and second interior pipe branch portions 120a, 120b.

Further, referring to FIGS. 5 and 6, the first and second exterior pipe branch portions 110a, 110b may include holding arm seating grooves 161 formed in interior surfaces of ends of the first and second exterior pipe branch portions 110a, 110b to receive the first and second holding arms 140a, 140b thereon. The holding arm seating grooves 161 may be connected to the interior pipe guiding grooves 151.

Hereinbelow, the use of the working tower 100 according to the first embodiment of the invention will be explained in greater detail with reference to FIGS. 3 and 4.

First, when the male screw 131 of the driving sleeve 130 is placed on the threadless section 124 formed between the first and second female screws 123, 122, it is possible to insert or withdraw the screw holder 140 to or from the exterior pipe 110 by a user (or auxiliary tool) by pulling or pushing the interior pipe 120. For reference, if the working tower 100 is used for minimally invasive spinal surgery, the user of the working tower 100 according to the first embodiment of the invention may be a surgeon.

In the state explained above, referring to FIG. 3, as the user (or auxiliary tool) pushes the interior pipe 120, the interior pipe 120 is moved in a downward direction, according to which the second female screw 122 of the interior pipe 120 is locked at a lower end of the driving sleeve 130. The screw holder 140 is completely withdrawn from the exterior pipe 110.

Referring to FIG. 4, when the user (or auxiliary tool) pulls the interior pipe 120, the interior pipe 120 is moved to an upward direction, according to which the second female screw 122 of the interior pipe 120 is locked at the male screw 131 of the driving sleeve 130. After that, when the user (or auxiliary tool) couples the male screw 131 to the second female screw 122 of the interior pipe 120 by rotating the driving sleeve 130, the screw holder 140 is completely withdrawn from the exterior pipe 110.

The rod inserter according to the second embodiment of the invention will be explained in greater detail below with reference to FIGS. 7 to 12.

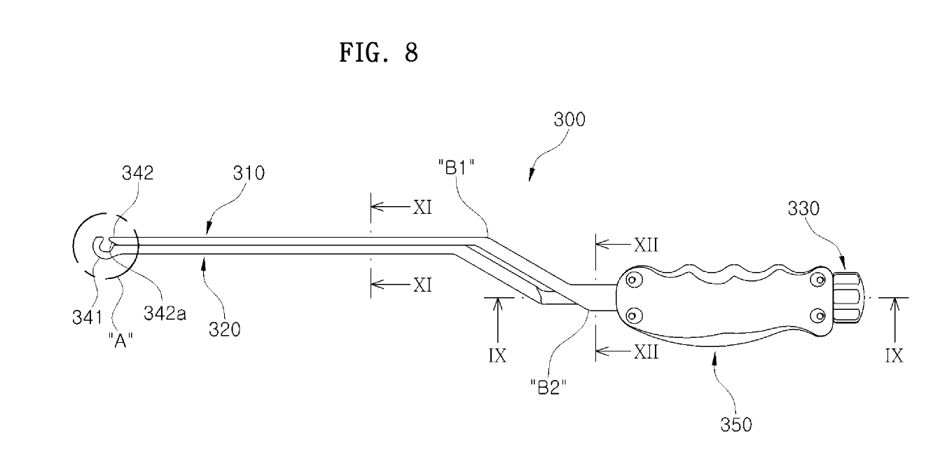

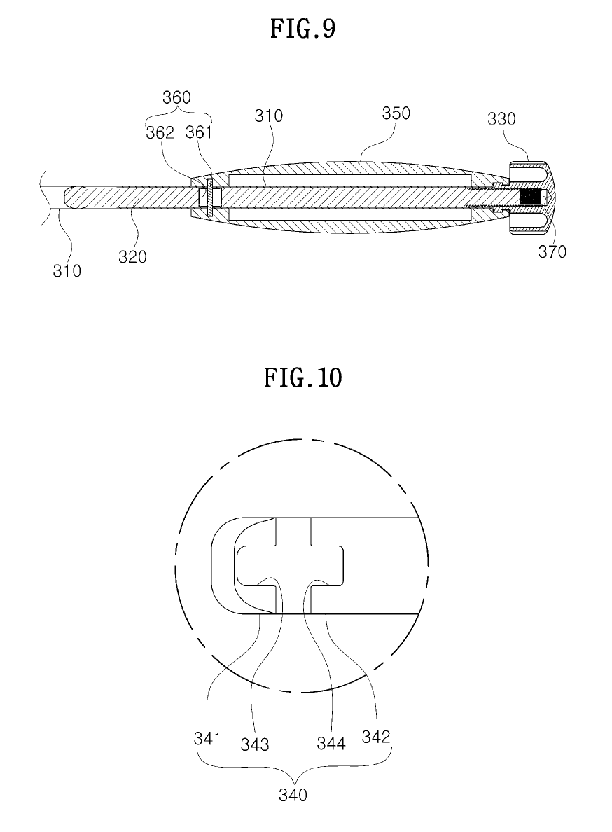

FIG. 7 is a perspective view of a rod inserter according to a second embodiment of the invention, FIG. 8 is a front view of the rod inserter of FIG. 7, and FIG. 9 is a cross section view taken on line IX-IX of the rod inserter of FIG. 8.

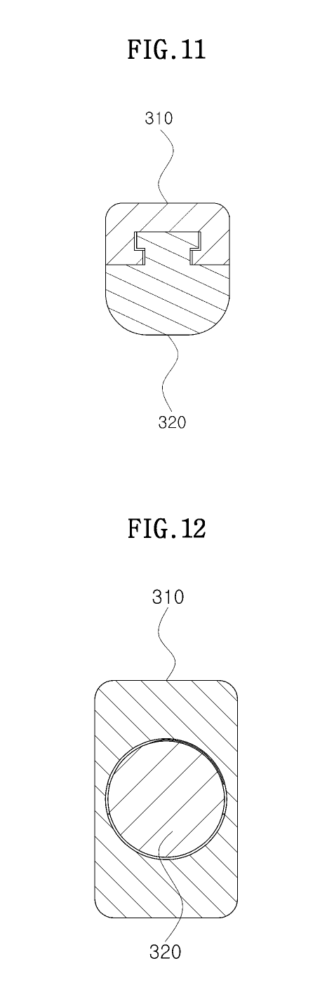

FIG. 10 is a plan view of encircled portion “A” of FIG. 8, FIG. 11 is a cross-section view taken on line XI-XI of the rod inserter of FIG. 8, and FIG. 12 is a cross-section view taken on line XII-XII of the rod inserter of FIG. 8.

According to the second embodiment of the invention, the rod inserter 300 grips the rod 20 and seats the rod 20 onto the rod accommodating part 11 of at least two correction screws 10. Referring to FIGS. 7 to 12, the rod inserter 300 includes a fixed bar 310, a moving bar 320, an adjusting knob 330, and a rod holder 340.

The fixed bar 310 is provided in the form of a bar which is fixed in relative to the moving bar 320, and slidably supports the moving bar 320 in the lengthwise direction thereof. Further, a fixed handle 350 may be provided on an exterior surface of the fixed bar 310 so that a user can easily hold the rod inserter 300. Referring to FIG. 7, one end of the fixed bar 310 corresponds to a left-hand side of the fixed bar 310, and the other end of the fixed bar 310 corresponds to the righthand side of the fixed bar 310.

The moving bar 320 is provided in the form of a bar which is moveable with respect to the fixed bar 310, and is slidably provided on the fixed bar 310 to slid along a lengthwise direction thereof. Referring to FIG. 7, one end of the moving bar 320 corresponds to a left-hand side of the moving bar 320, and the other end of the moving bar 320 corresponds to the righthand side of the moving bar 320.

The adjusting knob 330 adjusts the moving distance of the moving bar 320 with the rotation thereof. The adjusting knob 330 is rotatably connected to the fixed bar 310 and screw-coupled to the moving bar 320. Furthermore, a spacing portion 370 for moving, which is a hollow space, may be formed between the adjusting knob 330 and the other end of the moving bar 320 to allow the moving bar 320 to move toward the adjusting knob 330 sufficiently. Accordingly, because the moving bar 320 can be sufficiently moved toward the adjusting knob 330 due to the presence of the spacing portion 370 for moving, the gripping force of the rod holder 340 can be increased.

Referring to FIGS. 7 and 8, the rod holder 340 is provided between one end of the fixed bar 310 and one end of the moving bar 320 to grip the rod 20 in accordance with the moving of the moving bar 320. By way of example, the rod holder 340 may include a hook 341 provided on one end of the moving bar 320, and a rod supporting portion 342 provided on one end of the fixed bar 310. Further, the rod supporting portion 342 may have an arc-shaped contact surface 342a to grip a central portion of the rod 20 in cooperation with the hook 341 when the hook 341 is moved toward the adjusting knob 330. The rod holder 340 for gripping the central portion of the rod 20 will be explained below.

Referring to FIG. 10, the rod holder 340 may additionally include a first holding recess 343 formed in an end of the rod supporting portion 342, and a second holding recess 344 formed in one end of the hook 341 to grip an end of the rod 20 in cooperation with the first holding recess 343. The rod holder 340 for gripping an end of the rod 20 will be explained below.

Meanwhile, the moving bar 320 and the fixed bar 310 may have a first and a second bent portions B1, B2 to prevent interference with the working tower 100 (FIG. 4) and also to secure a clear view when the user inserts the rod 20 to the rod guiding groove 111 (FIG. 4) of the working tower 100 (FIG. 4) and the rod accommodating part 11 of the correction screw 10 in sequential order.

In the above-explained example, the fixing handle 350 and the rod holder 340 may be placed on parallel straight lines to enable the moving bar 320 to move efficiently along the lengthwise direction thereof. Hereinbelow, the structure that enables the moving bar 320 having the first and second bent portions B1, B2 on the fixed bar 310 will be explained with reference to FIGS. 8, 11 and 12.

Referring to FIGS. 8 and 11, the moving bar 320 and the fixed bar 310 may be slidably engaged with each other along a rail structure (FIG. 11) between the rod holder 340 and the first bent portion B1. Referring to FIGS. 8 and 12, an insertion structure (FIG. 12) may also be provided between the second bent portion B2 and the adjusting knob 330 to enable the moving bar 320 to slid along the fixed bar 310. Referring to FIG. 8, the moving bar 320 and the fixed bar 310 may be spaced apart from each other between the first and second bent portions B1, B2. Accordingly, the moving bar 320 may be provided to slid along the fixed bar 310 in a lengthwise direction thereof due to the rail structure (FIG. 11), the insertion structure (FIG. 12) and the spacing structure.

Further, referring to FIG. 9, the moving bar 320 may be connected to the fixed bar 310 via a first movement restricting unit 360 to restrict the moving distance of the moving bar 320. By way of example, the first movement restricting unit 360 may include a stop pin 361 and a movement restricting groove 362. The stop pin 361 is provided across the fixed bar 310. The movement restricting groove 362 is formed in the moving bar 320 to receive the stop pin 361 therein. The movement restricting groove 362 is an elongated hole with one and the other ends, in which the stop pin 361 is inserted and locked at the stop pin 361 according to the movement of the moving bar 320.

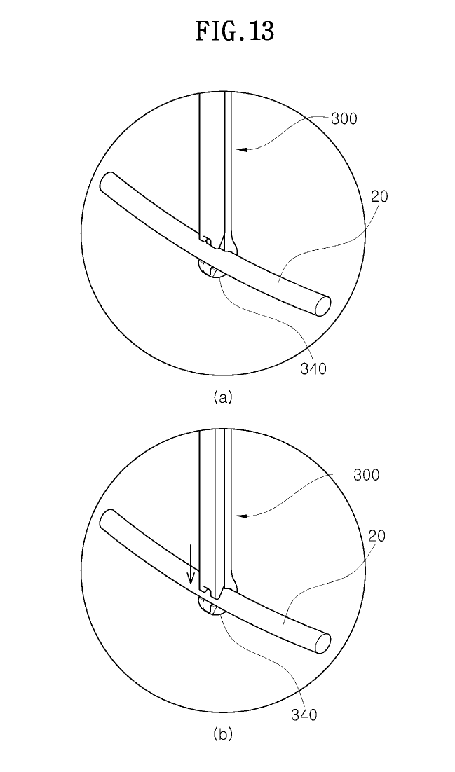

Hereinbelow, the use of the rod inserter according to the second embodiment of the invention will be explained in greater detail with reference to FIGS. 13A to 16.

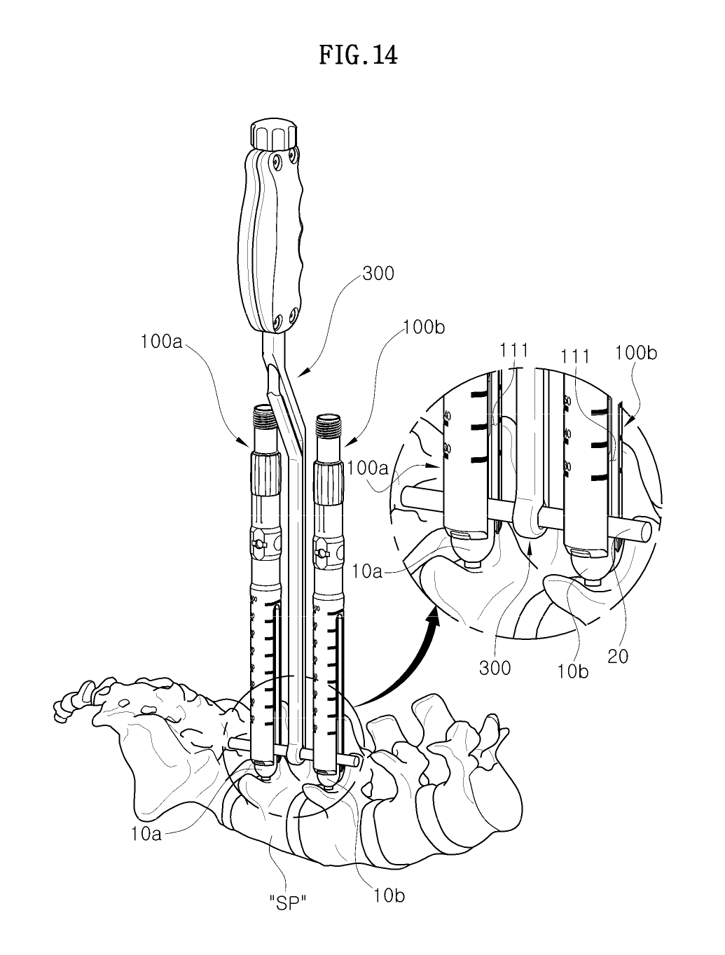

FIGS. 13A and 13B are views illustrating the states before and after the rod inserter 300 of FIG. 7 grips a center of a rod 20, and FIG. 14 is a schematic perspective view illustrating the rod inserter 300 of FIG. 7, gripping the center of the rod 20, inserting the rod 20 into a rod guiding groove 111 of FIG. 4 of the working tower 100 of FIG. 4.

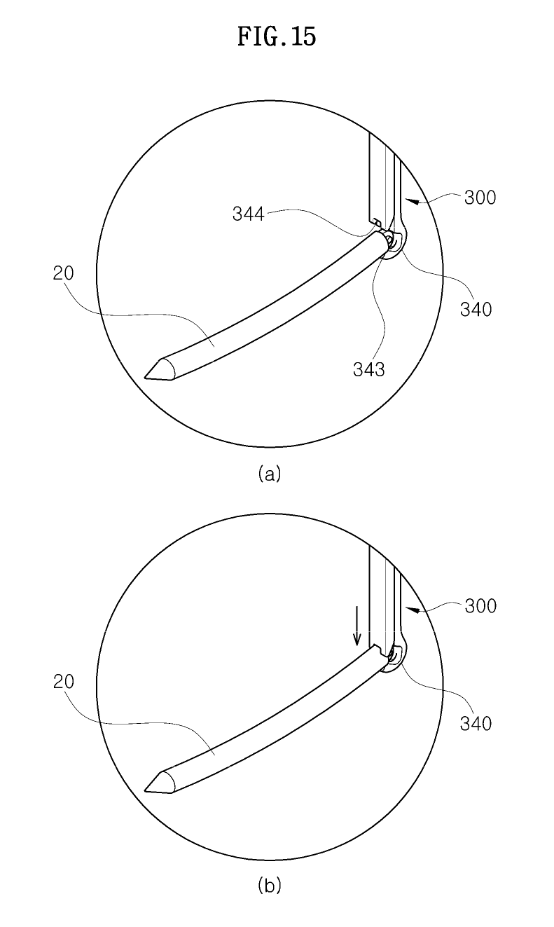

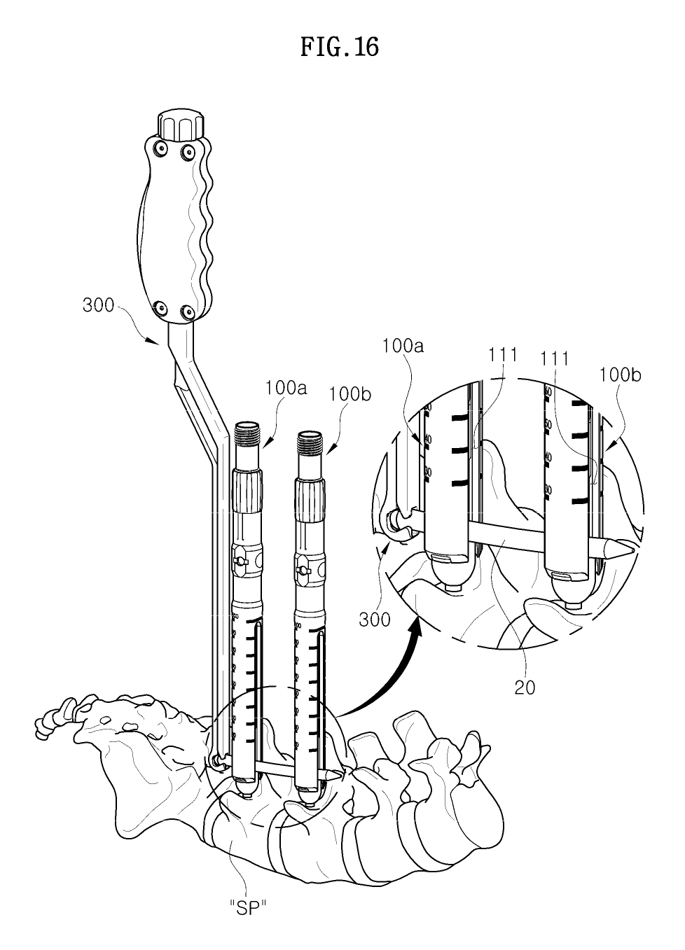

FIGS. 15A and 15B are views illustrating the states before and after the rod inserter 300 of FIG. 7 grips the rod end, and FIG. 16 is a schematic perspective view illustrating the rod inserter 300 of FIG. 7, gripping the end of the rod 20, inserting the rod into a rod guiding groove 111 of FIG. 4 of the working tower 100 of FIG. 4.

Referring to FIG. 13A, in one embodiment, the center of the rod 20 is placed across the rod holder 340 and the user rotates the adjusting knob 330 in a forward direction to move the moving bar 320 toward the adjusting knob 330 as much as possible, according to which, referring to FIG. 13B, the center of the rod 20 is gripped by the hook 341 and the rod supporting portion 342. For reference, if the rod inserter 300 is used for minimally invasive spinal surgery, the user of the rod inserter 300 according to the second embodiment of the invention may be a surgeon.

Referring to FIG. 14, with the center of the rod 20 gripped as explained above, and with the rod inserter 300 placed between a first and a second working towers 100a, 100b, one end of the rod 20 is first inserted by the rod inserter 300 into the rod guiding groove 111 of the first working tower 100a. The rod inserter 300 is then turned so that the other end of the rod 20 is inserted into the rod guiding groove 111 of the second working tower 100b. Next, the rod 20 is descended by the rod inserter 300 so that both ends of the rod 20 are seated in the rod accommodating part 11 of the first and second correction screws 10a, 10b. Lastly, when operation using a rod inducer 700 (FIG. 27, to be explained below) is completed, the adjusting knob 330 is rotated in a reverse direction to ungrip the rod 20.

Meanwhile, referring to FIG. 15A, the end of the rod 20 may be gripped according to one embodiment in which the end of the rod 20 is placed to face the rod holder 340, and the user rotates the adjusting knob 330 to a forward direction to move the adjusting bar 320 toward the adjusting knob 330 as much as possible, referring to FIG. 15B, the end of the rod 20 is gripped by the first and second holding recesses 343, 344.

Referring to FIG. 16, with the end of the rod 20 being gripped as explained above, and with the rod inserter 300 being placed on one side of the first working tower 100a opposite to a side facing the second working tower 100b, one end of the rod 20 is inserted into the rod guiding groove 111 of the first working tower 100a and the rod guiding groove 111 of the second working tower 100b in sequence by the rod inserter 300. Next, the rod 20 is descended by the rod inserter 300 so that both ends of the rod 20 are seated in the rod accommodating part 11 of the first and second correction screws 10a, 10b. Lastly, when operation using a rod inducer 700 (FIG. 27, to be explained below) is completed, the adjusting knob 330 is rotated in a reverse direction to ungrip the rod 20.

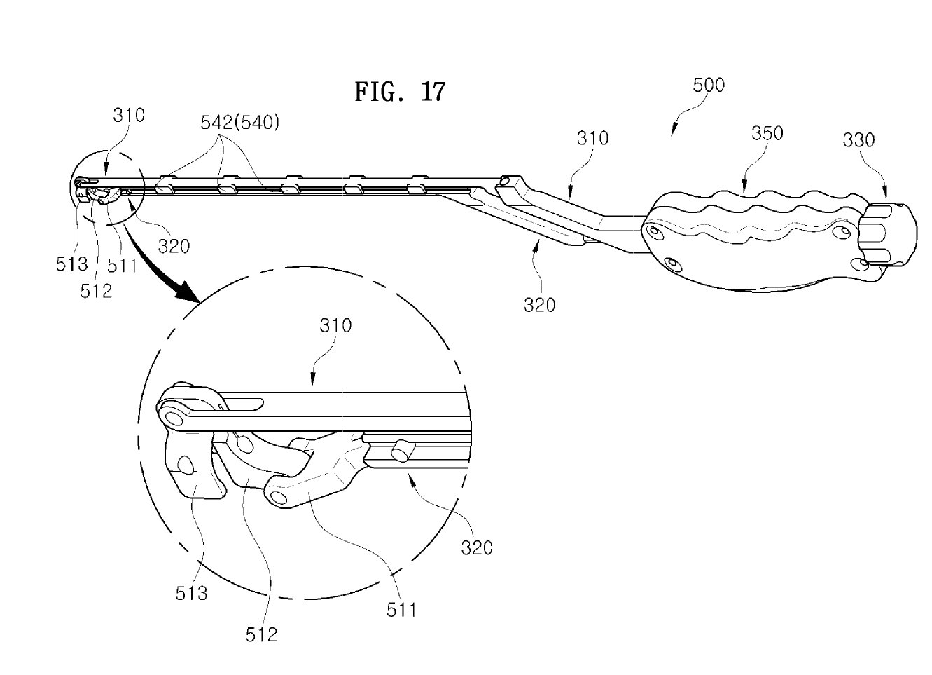

The rod inserter according to a modified example of the second embodiment of the invention will be explained in greater detail below with reference to FIGS. 17 to 21.

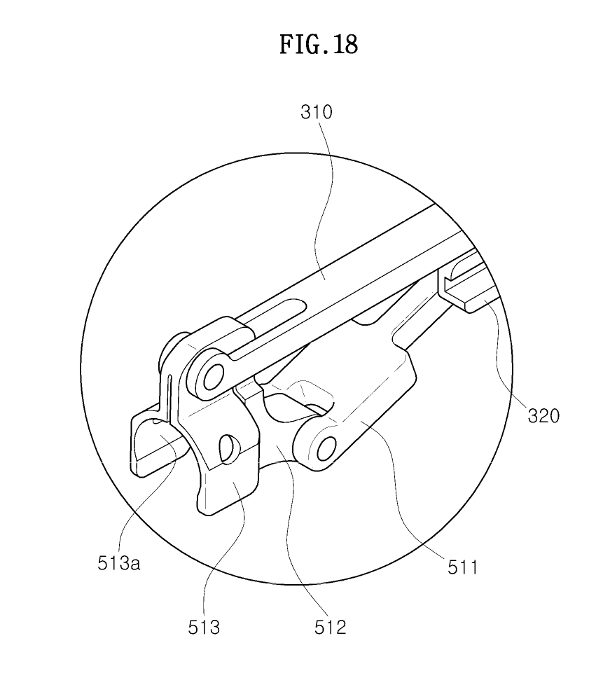

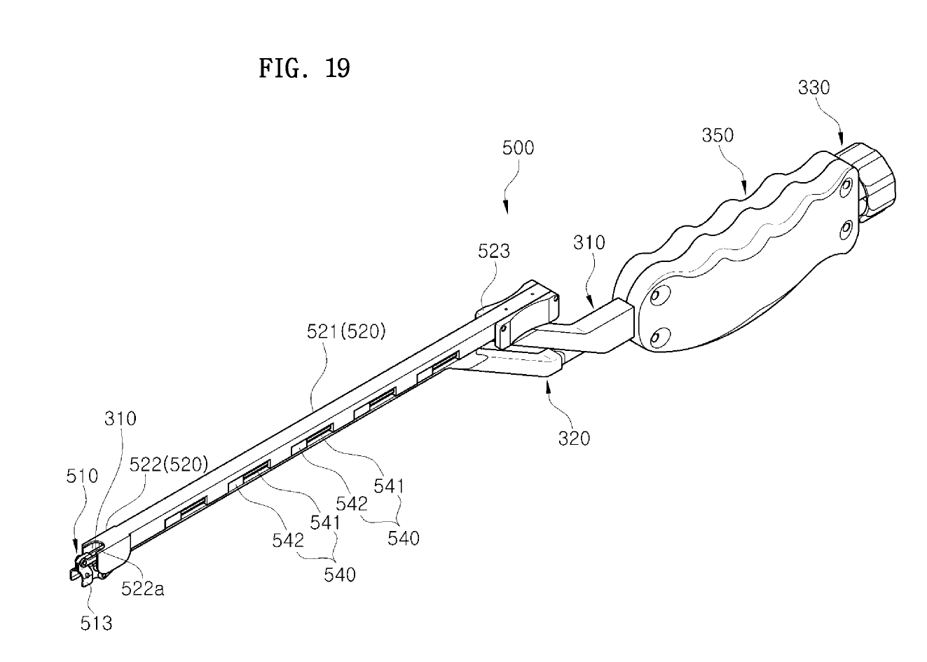

FIG. 17 is a front view illustrating a rod inserter according to a modified example of the second embodiment of the invention, FIG. 18 is a perspective view illustrating a rod holder of the rod inserter of FIG. 17, and FIG. 19 is a perspective view illustrating a cover being mounted on the rod inserter of FIG. 17.

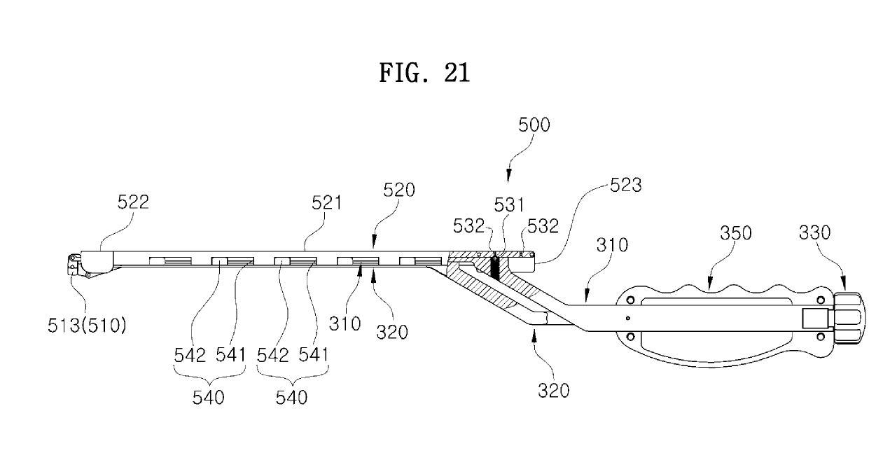

FIG. 20 is a front view of the rod inserter of FIG. 19, and FIG. 21 is a cutaway view illustrating a main portion of the rod inserter of FIG. 20.

Referring to FIGS. 17 to 21, a rod inserter 500 according to a modified example of the second embodiment of the invention is almost identical to that of the second embodiment explained above, except for the changes in the constitution of the rod holder 510, addition of a cover 520 and addition of a first multi-step adjusting unit 530 and a second movement restricting unit 540. Accordingly, only the rod holder 510, the cover 520, the first multi-step adjusting unit 530 and the second movement restricting unit 540 will be explained below. Further, the same reference numerals will be used for the like elements of the second embodiment of the invention.

Referring to FIGS. 17 and 18, the rod holder 510 includes a knuckle 511, a rod holder bracket 512 and a clip 513.

The knuckle 511 is rotatably connected between components to increase freedom of rotation. One end of the knuckle 511 is rotatably provided on one end of the moving bar 320 and the other end thereof is rotatably provided on one end of the rod holder bracket 512. Referring to FIG. 17, one end of the knuckle 511 corresponds to the righthand side of the knuckle 511, while the other end corresponds to the left-hand side of the knuckle 511.

The rod holder bracket 512 is provided in the bent form and to fix the clip 513. One end of the rod holder bracket 512 is rotatably provided on the other end of the knuckle 511, while the other end of the rod holder bracket 512 is rotatably provided on one end of the fixed bar 310. Referring to FIG. 17, one end of the rod holder bracket 512 corresponds to the righthand side of the rod holder bracket 512, while the other end corresponds to the left-hand side of the rod holder bracket 512. Referring also to FIG. 17, the other end of the rod holder bracket 512 is bent approximately in an upward direction.

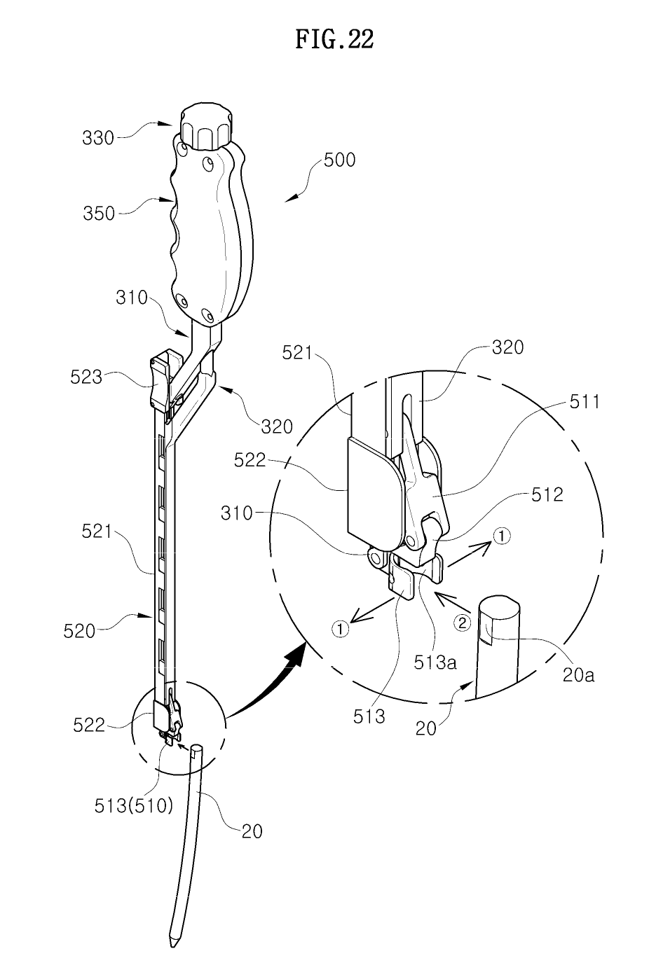

The clip 513 is fixed to the rod holder bracket 512 and grips an end of the rod 20. To be specific, referring to FIG. 18, the clip 513 may be provided in the arc form. Both ends of the clip 513 may be elastically spread from each other to efficiently receive an end of the rod 20 in the clip 513. Further, a coupling portion 513a may be formed on an interior surface of the clip 513 to grip the end of the rod 20. By way of example, if a coupling recess 20a (FIG. 22) is formed in the end of the rod 20, the coupling portion 513a may be provided as a coupling protrusion. Alternatively, and although not illustrated herein, if a coupling protrusion (not illustrated) is formed on the end of the rod 20, a corresponding coupling recess (not illustrated) may be formed in the end of the rod 20.

Due to the constitution of the rod holder 510 as explained above, when the moving bar 320 moves towards the knuckle 511, the rod holder bracket 512 and the clip 513 are rotated by the knuckle 511 with respect to one end of the fixed bar 310 so that the rod 20 gripped at the clip 513 is rotated together. This will be explained in greater detail below.

Referring to FIGS. 19 to 21, a cover 520 may be slidably provided on an exterior surface of the moving bar 320 to slide in a lengthwise direction thereof. The cover 520 may particularly include a cover body 521 to cover the fixed bar 310, and a clip locking portion 522 to cover the clip 513 in a direction the cover 520 faces the clip 513 to prevent splitting of the clip 513. Accordingly, because the clip 513 is perfectly gripped by the clip locking portion 522 of the cover 520, the rod 20 is prevented from being released from the clip 513 due to unintended splitting thereof, once the rod 20 is gripped at the clip 513.

Further, a rod passing hole 522a may be formed in the clip locking portion 522 to allow efficient rotation of the rod 20 without being interfered by the clip locking portion 522, when the moving bar 320 is moved towards the clip 513 after the clip 513 is locked at the clip locking portion 522.

Furthermore, the cover 520 may include a cover handle 523 provided on the other end of the cover body 521 to enable the user to push or pull the cover 520 with ease.

Referring to FIG. 21, the cover 520 may be connected to the fixed bar 310 via the first multi-step adjusting unit 530 to provide the user with the operation feeling when the cover 520 is slid. By way of example, the first multi-step adjusting unit 530 may include a first ball 531 elastically supported on the fixed bar 310, and a plurality of first locking holes 532 formed in the cover body 521 so that the first ball 531 is locked in multiple steps. The “clicking” sound is generated when the first ball 531 is located in the first locking holes 532 or separated therefrom, which can give the user with the maximized operation feeling.

Referring to FIGS. 20 and 21, the cover 520 may be connected to the fixed bar 310 through a second movement restricting unit 540 to restrict a moving distance of the cover 520. By way of example, the second movement restricting unit 540 may include at least one long hole 541 formed in the cover along a lengthwise direction thereof, and at least one stop protrusion 542 formed on the fixed bar 310 to move in said at least one long hole 541.

The use of the rod inserter 500 according to a modified example of the second embodiment of the invention will be explained below with reference to FIGS. 22 to 25.

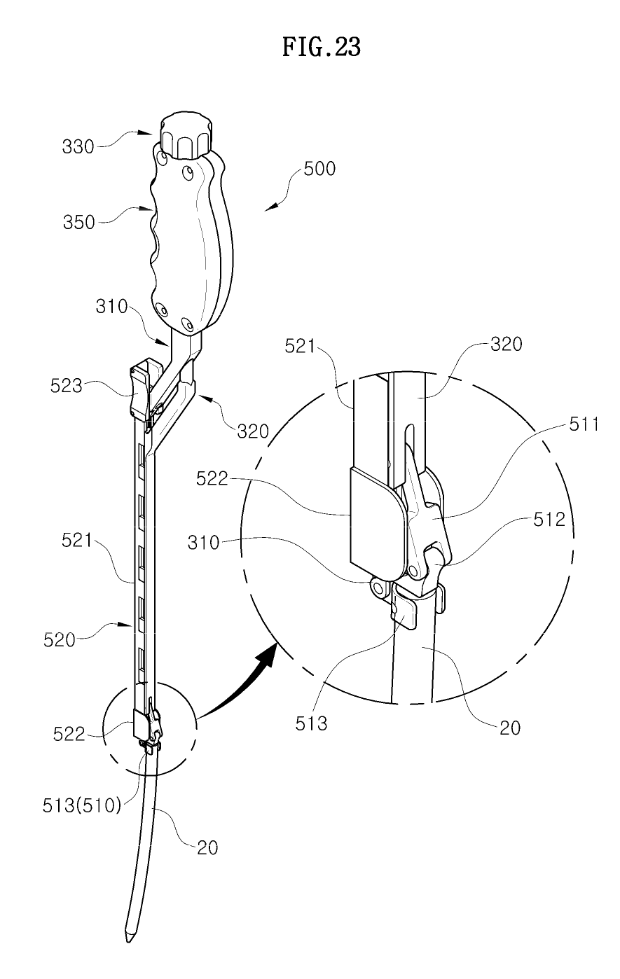

FIG. 22 is a perspective view illustrating a rod 20 being mounted on the rod inserter 500 of FIG. 19, and FIG. 23 is a perspective view illustrating a rod 20 after mounted on the rod inserter 500 of FIG. 19.

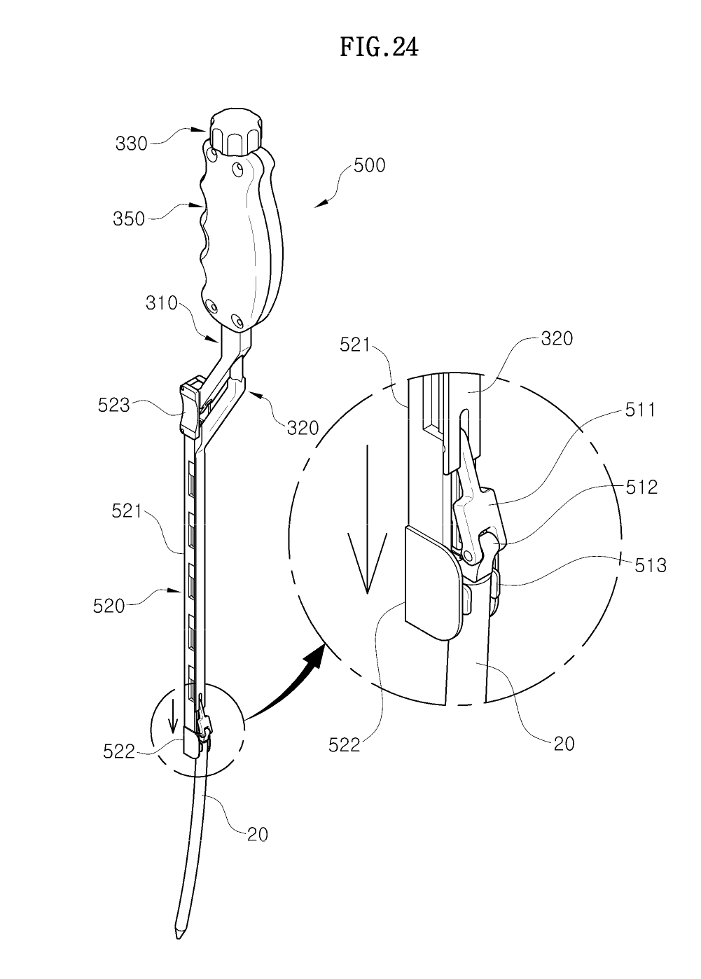

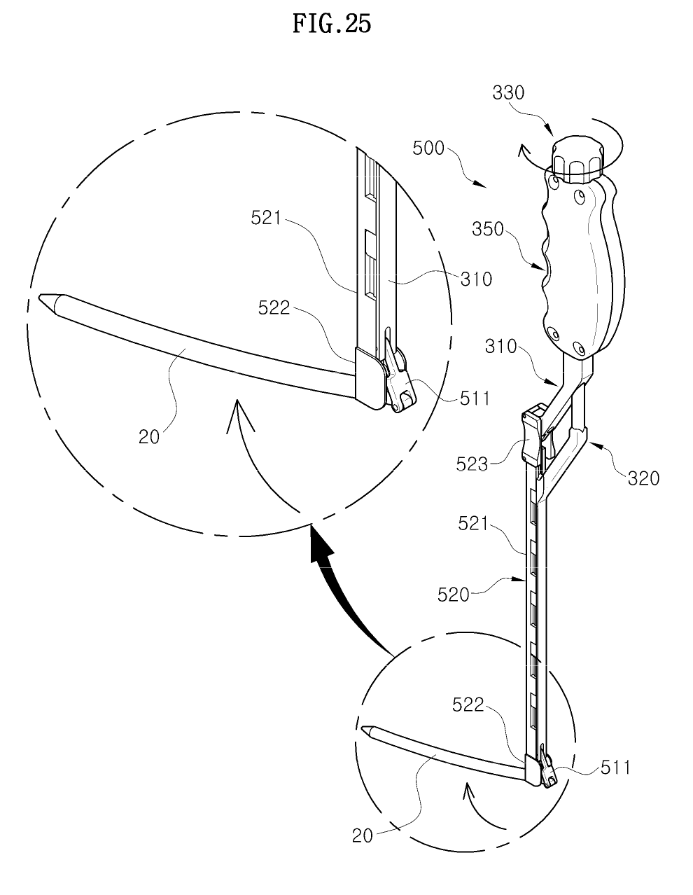

FIG. 24 is a perspective view illustrating the cover 520 of the rod inserter 500 moving from the state of FIG. 23 toward the clip 513, causing the clip 513 locked, and FIG. 25 is a perspective view illustrating a rod 20 being rotated as a moving bar 320 is moved toward the clip 513 in accordance with rotation of an adjusting knob 330 in the state of FIG. 24.

Referring first to FIG. 22, when the user moves the clip locking portion 522 of the cover 520 in an upward direction to thus expose the clip 513 of the rod holder 510 to outside, the clip 513 is elastically split apart in an arrowed direction 1. Referring to FIG. 23, the end of the rod 20 is placed in the clip 513. At this time, the coupling protrusion 513a of the clip 513 is loosely coupled to the coupling hole 20a in the end of the rod 20. For reference, if the rod inserter 500 according to the modified example of the second embodiment of the invention is used for spinal minimally invasive surgery, the user may be a surgeon.

Referring to FIG. 24, when the user moves the clip locking portion 522 of the cover 520 in a downward direction of the drawing, the clip 513 is tightened by the clip locking portion 522 so that the end of the rod 20 is perfectly mounted on the clip 513.

After that, referring to FIGS. 17, 18 and 25, when the user turns the adjusting knob 330 in the arrowed direction, the moving bar 320 is moved to the downward direction of the drawings, thereby pushing the knuckle 511 which in turn causes the rod holder bracket 512 and the clip 513 to rotate with respect to one end of the fixed bar 310. Accordingly, the rod 20 gripped by the clip 513 is also rotated and bent with respect to the moving bar 320.

For reference, the user places the rod inserter 500 on a side of the first working tower 100a (FIG. 16) opposite to a side facing the second working tower 100b (FIG. 16), in a state that the rod 20 in the state illustrated in FIG. 24 is not bent with respect to the moving bar 320. Then when the rod 20 is bent to the state illustrated in FIG. 25, the rod 20 is inserted into the rod guiding groove 111 (FIG. 16) of the first working tower 100b (FIG. 16) and the rod guiding groove 111 (FIG. 16) of the second working tower 100b (FIG. 16) in sequence. The user then descends the rod 20 with the rod inserter 500 to seat both ends of the rod 20 on the rod accommodating part 11 of the first and second correction screws 10a, 10b. Lastly, when the operation using the rod inducer 500 (to be explained below) is completed, the user releases the rod 20 from the gripped state by moving the clip locking portion 522 to the upward direction of the drawing. That rod 20 is un-gripped when the clip locking portion 522 is moved to the upward direction, because the clip 513 is elastically split apart.

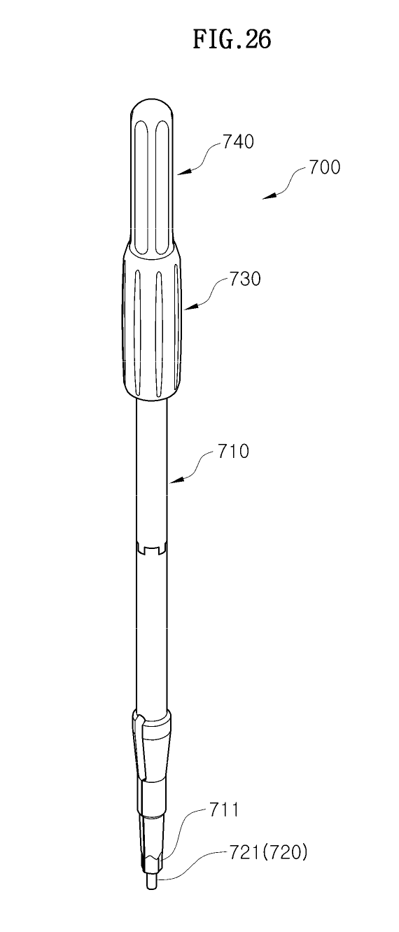

The rod reducer according to the third embodiment of the invention will be explained below with reference to FIGS. 26 and 27.

FIG. 26 is a perspective view of a rod reducer according to a third embodiment of the invention, and FIG. 27 is a longitudinal cross-section view of the rod reducer of FIG. 26.

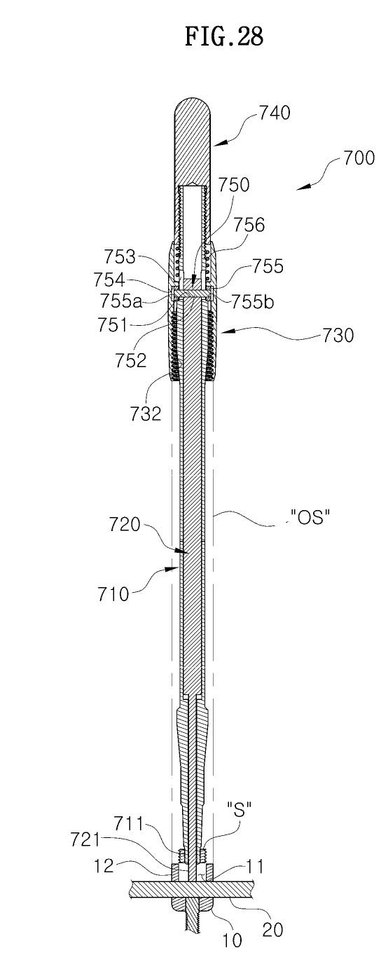

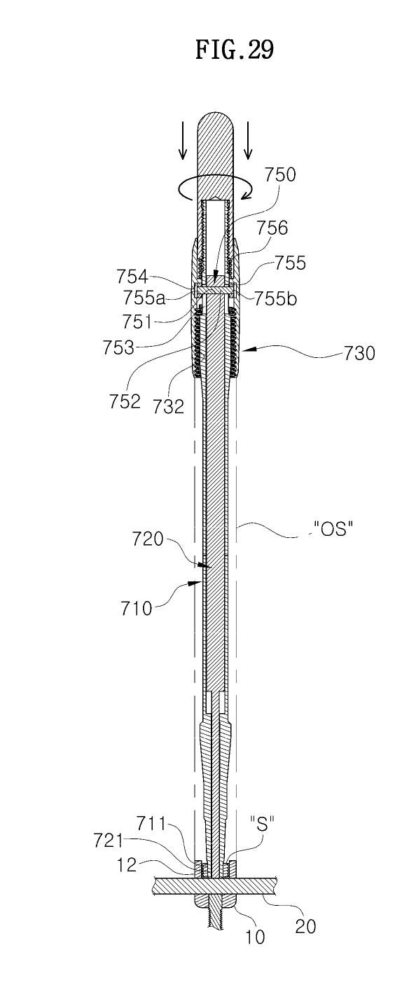

The rod reducer 700 according to the third embodiment of the invention presses the rod 20 (FIG. 28) seated on the rod accommodating part 11 (FIG. 28) of the correction screw 10 (FIG. 28) and at the same time couples a set screw S (FIG. 29) onto the rod accommodating part 11 (FIG. 29). Referring to FIGS. 26 and 27, the rod reducer 700 includes an exterior member 710, an interior member 720, a coupling sleeve 730, and a rotation handle 740.

The exterior member 710, provided in a tubular form, has a set screw mounting part 711 formed on a lower portion thereof to mount the set screw S (FIG. 28).

The interior member 720, provided in a rod-like form, is slidably formed inside the exterior member 710, and includes a lower withdrawing part 721 withdrawn from the exterior member 710 on a lower end thereof to contact the rod 20 (FIG. 28).

The coupling sleeve 730 causes the interior member 720 to descend so that the rod 20 (FIG. 28) is pressed by the lower withdrawing part 721 of the interior member 720 while the coupling sleeve 730 is coupled to a third external device (OS) (e.g., working tower 100 of FIG. 4) gripping the correction screw 10. If the third external device (OS) takes the shape of tubular form like the working tower 100, the exterior member 710 may have a structure for insertion into the third external device (see FIG. 28).

The rotation handle 740 rotates the exterior member 710 so that the set screw S (FIG. 29) is screw-coupled to the rod accommodating part 11 (FIG. 29) of the correction screw 10 (FIG. 29). The rotation handle 740 may be securely fixed to the upper end of the exterior member 710.

Furthermore, an inner circumference of the coupling sleeve 730 may have a structure for screw-coupling with an external circumference of the third external device OS (FIG. 28). Referring to FIG. 27, a male screw 732 for external device (“second male screw”) may be formed on the inner circumference of the coupling sleeve 730. By way of example, if the third external device OS is the working tower 100 (FIG. 4), the second male screw 732 may be screw-coupled with the first female screw 123 (FIG. 4) of the working tower 100.

Further, referring to FIG. 27, the coupling sleeve 730 may be connected to the exterior member 710 and the interior member 720 by a connect unit 750. By way of example, the connect unit 750 may include a piercing hole 751 formed through the interior member 720, a connect pin 752 inserted into the piercing hole 751, a sliding recess 753 formed in the exterior member 710 to receive therein both ends of the connect pin 752, the sliding recess 753 being elongated circumferentially in the inner circumference of the coupling sleeve 730, a mount recess 754 formed along a circumferential direction on an inner circumference of the coupling sleeve 730, and a rotation ring 755 rotatably mounted on the mount recess 754, the rotation ring 7555 in which a first and a second insertion holes 755a, 755b are formed to receive therein both ends of the connect pin 752.

The sliding recess 753 may be set to have a longer length than that of the lower withdrawing part 721 of the interior member 720 with reference to the lengthwise direction of the exterior member 710 so that the lower withdrawing part 721 of the interior member 720 is completely inserted into the exterior member 710 when the set screw S is coupled to the rod accommodating part 11 of the correction screw 10.

Further, the interior member 720 may be elastically supported on the exterior member so that the lower withdrawing part 721 of the interior member 720 is maintained in a withdrawn state from the exterior member 710 as long as the set screw S (FIG. 29) is not coupled to the rod accommodating part 11 (FIG. 29) of the correction screw 10 (FIG. 29). By way of example, an elastic member 756 may be provided between the coupling sleeve 730 and the rotation handle 740 for elastic support, so that the interior and exterior members 720, 710 are elastically supported on each other via the coupling sleeve 730 and the rotation handle 740, respectively.

The use of the rod reducer 700 according to the third embodiment of the invention will be explained in greater detail below with reference to FIGS. 28 and 29.

FIG. 28 is a schematic cross-section view illustrating the rod 20 being pressed by the lower withdrawing part 721 of the interior member 720 while the coupling sleeve 730 is being rotated.

FIG. 29 is a schematic cross-section view illustrating a set screw S being coupled into the rod inserting portion 11 of the correction screw 10 while the rotation handle is being pressed and rotated.

First, the user inserts the rod reducer 700 into the third external device (OS). For reference, if the rod reducer 700 is used for minimally invasive spinal surgery, the user of the rod reducer 700 according to the third embodiment of the invention may be a surgeon.

After that, referring to FIG. 28, as the user rotates the coupling sleeve 730, the coupling sleeve 730 is efficiently rotated by the rotation ring 755 and at the same time, the second male screw 732 of the coupling sleeve 730 is screw-coupled with the third external device OS (e.g., first female screw 123 of FIG. 4 of the working tower 100 of FIG. 4), so that the coupling sleeve 70 is moved downward. At this time, both the exterior and interior members 710, 720 secured with the coupling sleeve 730 by the connect pin 752 are also moved downward.

When the rod 20 is brought into contact with the bottom surface of the rod accommodating part 11 of the correction screw 10 by the lower withdrawing part 721 of the interior member 720, referring to FIG. 29, the user stops rotating the coupling sleeve 730, presses the rotation handle 740 in a downward direction and also rotates the rotation handle 740 in a forward direction so that the set screw S is screw-coupled with the rod accommodating part 11 of the correction screw 10. At this time, the interior member 720 is fixed with the coupling sleeve 730, while only the exterior member 710 is moved downward through the sliding recess 753. That is, the interior member 720 is gradually entered into the exterior member 710 while the exterior member 710 is gradually moved in the downward direction.

Accordingly, since both pressing on the rod 20 and coupling of the set screw S are performed at the same time, the chances that the tissue is caught between the rod 20 and the set screw S are prevented.

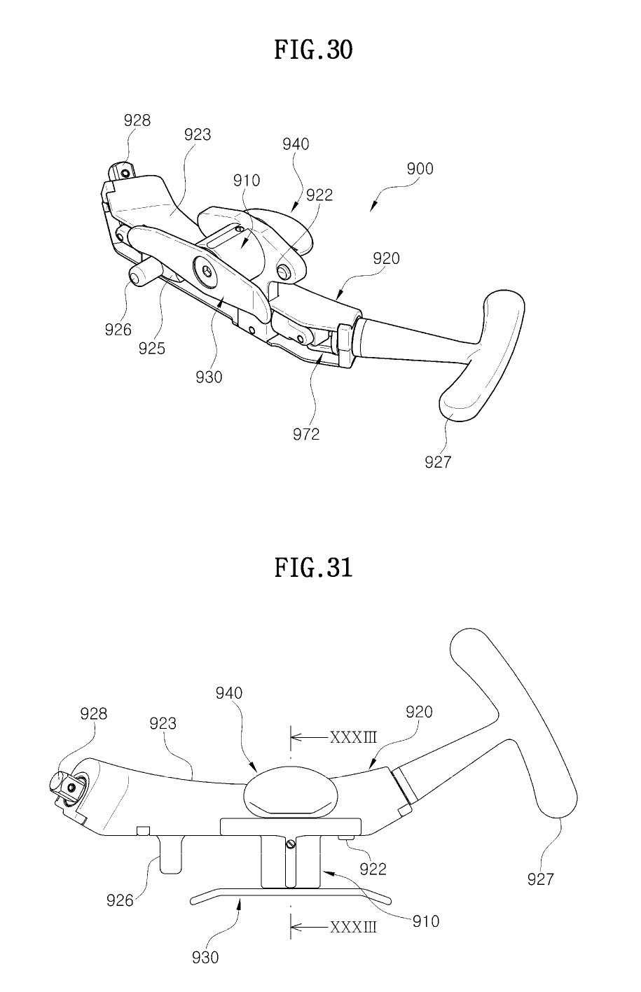

The compression-distraction tool according to the fourth embodiment of the invention will be explained in greater detail below with reference to FIGS. 30 to 35.

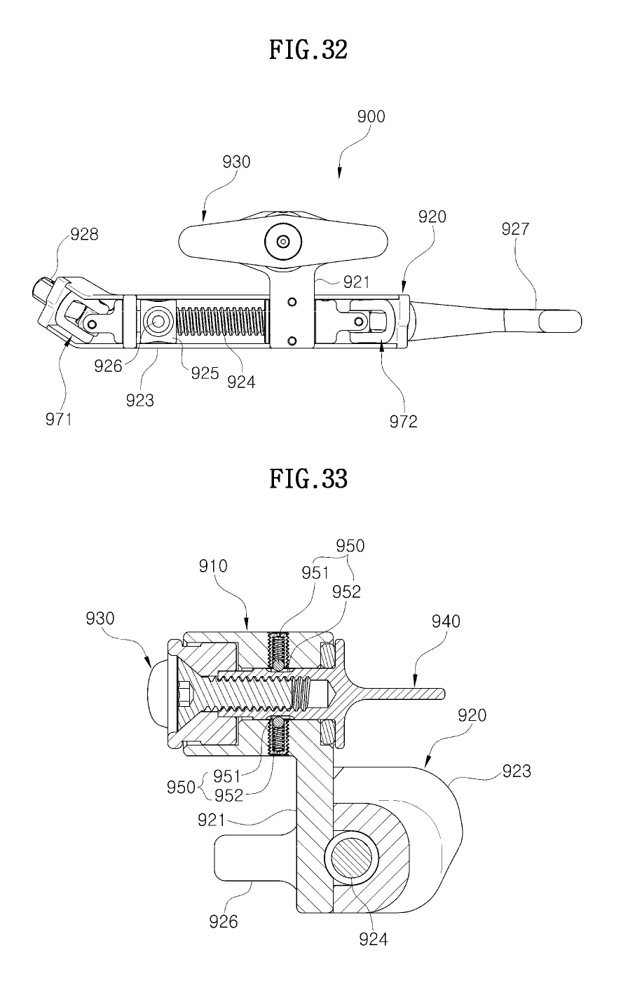

FIG. 30 is a perspective view of a compression-distraction tool according to a fourth embodiment of the invention, FIG. 31 is a plan view of the compression-distraction tool of FIG. 30, and FIG. 32 is a front view of the compression-distraction tool of FIG. 30.

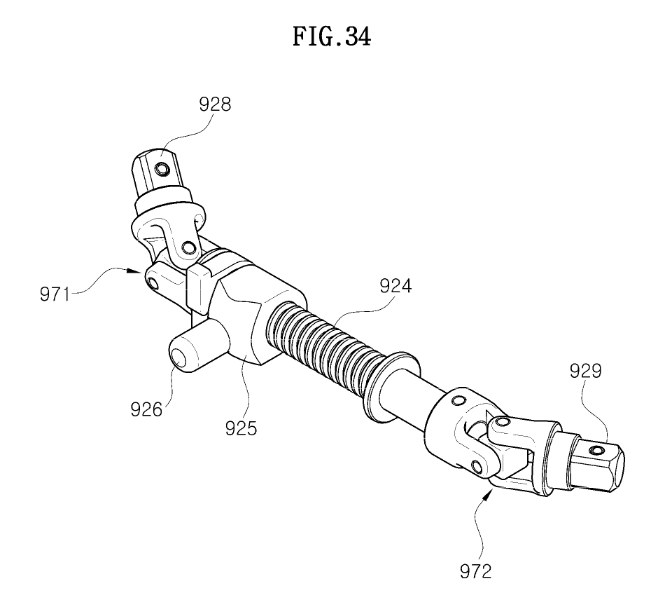

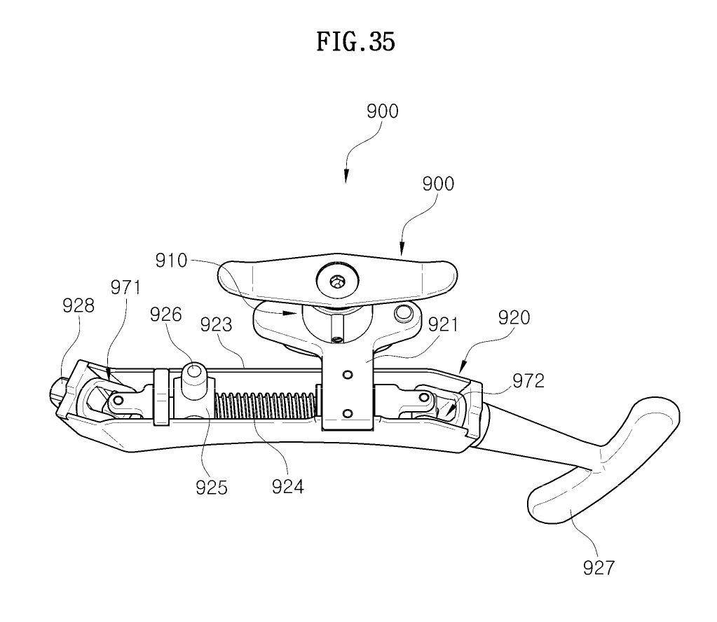

FIG. 33 is a cross-section view taken on line XXXIII-XXXIII of the compression-distraction tool of FIG. 31, FIG. 34 is a detailed perspective view of a screw rod or the like of the compression-distraction tool of FIG. 32, and FIG. 35 is a perspective view of the compression-distraction tool of FIG. 30 viewed from a front upper direction.

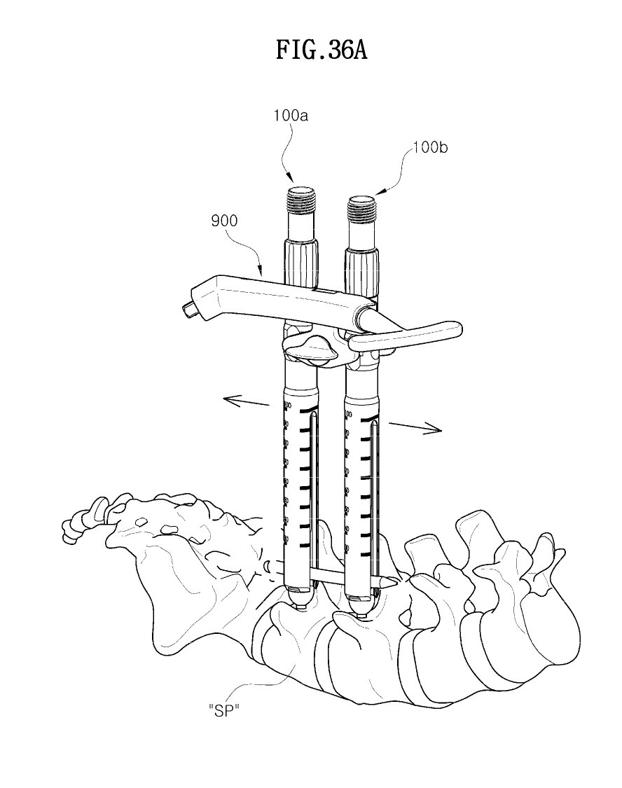

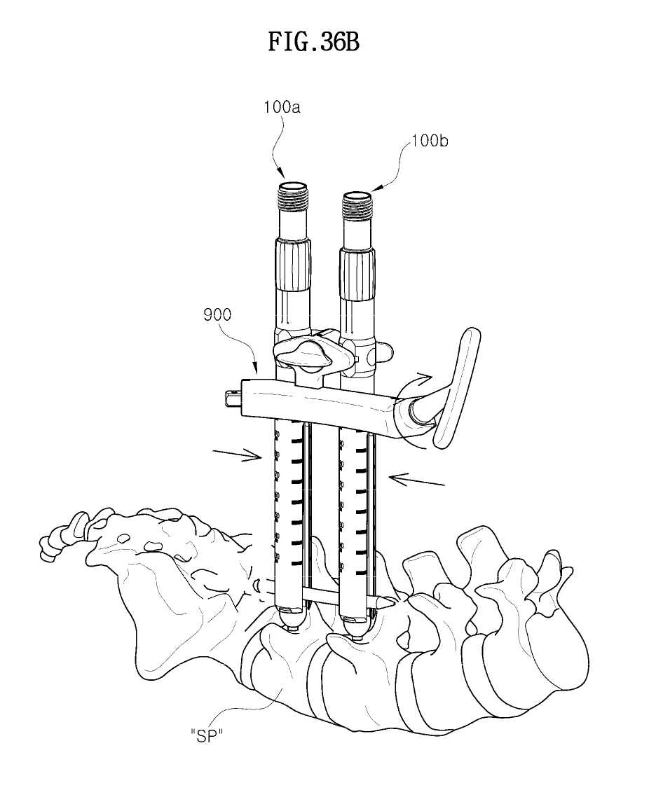

The compression-distraction tool 900 according to the fourth embodiment of the invention performs either distraction by spreading a distance between the first and second correction screws 10a, 10b (FIG. 36A) on lower ends of the first and second working towers 100a, 100b (FIG. 36a), or compression by reducing the distance. Referring to FIGS. 30 to 35, the compression-distraction tool 900 includes a pivot 910, a tower compressing unit 920 and a tower supporter 930.

The pivot 910 takes the form of a cylindrical column and is positioned approximately vertically between the first and second working towers 100a, 100b.

The tower compressing unit 920 is provided on one end of the pivot 910 and compresses the first and second working towers 100a, 100b to tilt with respect to the pivot 910. As used herein, the one end of the pivot 910 corresponds an upper end of the pivot in FIG. 31.

The tower supporter 930 is rotatably provided on the other end of the pivot 910 to prevent sliding movement of the first and second working towers 100a, 100b in a lengthwise direction of the pivot 910. As used herein, the other end of the pivot 910 corresponds to a lower end of the pivot 910 in FIG. 31.

Furthermore, the compression-distraction tool 900 according to the fourth embodiment of the invention may additionally include an adjusting handle 940 to rotate the tower supporter 930 so that the tower supporter 930 enters in between the first and second working towers 100a, 100b. By way of example, the adjusting handle 940 may be fixed on the tower supporter 930 to be rotated about the pivot 910.

Referring to FIG. 33, the adjusting handle 940 may be connected to the pivot 910 via the second multi-step adjusting unit 950 to provide the user with the operation feeling when the adjusting handle 940 is rotated. By way of example, the second multi-step adjusting unit 950 may include a second ball 951 elastically supported on the pivot 910, and a plurality of second locking holes 952 formed in the outer circumference of the adjusting handle 940 so that the second ball 951 is locked in multiple steps. The “clicking” sound is generated when the second ball 951 is located in the second locking holes 532 or separated therefrom, which can give the user with the maximized operation feeling.

In one embodiment, four second locking holes 952 may be provided at 90° interval so that the tower supporter 930 is placed either approximately parallel with respect to the first and second working towers 100a, 100b to facilitate the insertion of the tower supporter 930 into the first and second working towers 100a, 100b, or approximately vertical with respect to the first and second working towers 100a, 100b to cause the tower supporter 930 to support the first and second working towers 100a, 100b.

Referring to FIGS. 32, 34 and 35, the tower compressing unit 920 may include a connect bracket 921 vertically provided on one end of the pivot 910, a fixation protrusion 922 provided on the connect bracket 921 to be fit into the external device insertion hole 113 (FIG. 4) of the first working tower 100a, a compressing body 923 provided on the connect bracket 921 at a predetermined distance from the pivot 910, a screw rod 924 rotatably provided on the compressing body 923, a compressing nut 925 screw-coupled with the screw rod 924, a compressing protrusion 926 provided on the compressing nut 925 and placed on one side of the second working tower 100b to pull the second working tower 100b, and a driving handle 927 rotatably provided on an exterior side of the compressing body 923 to rotate the screw rod 924 so that compressing nut 925 is moved. Accordingly, as the user rotates the driving handle 927 in a forward or reverse direction, the screw rod 924 is rotated in a forward or reverse direction, causing the compressing nut 925 to move in a leftward or rightward direction in the drawing. Accordingly, the compressing protrusion 926 provided on the compressing nut 925 also moves to securely grip the first and second working towers 100a, 100b in cooperation with the fixation protrusion 922.

Further, referring to FIGS. 30 and 35, the compressing body 923 is positioned vertically on the pivot 910 and the connect bracket 921, respectively, and may be elongated across the first and second working towers 100a, 100b (see FIG. 36A).

Further, referring to FIG. 31, both ends of the compressing body 923 may be bent into a direction different from the direction toward the first and second working towers 100a, 100b to minimize interference with the first or second working tower 100a, 100b when the user rotates the driving handle 927.

Further, referring to FIGS. 32, 34 and 35, for efficient rotation of the screw rod 924, both ends of the screw rod 924 may be connected to both ends of the compressing body 923, which are in bent shapes, by a first and a second universal joints 971, 972, respectively.

Further, the tower compressing unit 920 may additionally include a first and a second sub-shafts 928, 929 provided rotatably on both outer ends of the compressing body 923, and the driving handle 927 may be coupled to either one of the first and second sub-shafts 928, 929 to rotate the screw rod 924. Accordingly, irrespective of whether the pivot 910 is placed above or below the compressing body 923, a right-handed user or a left-handed user may conveniently operate the tower compressing unit 920.

The use of the compression-distraction tool 900 according to the fourth embodiment of the invention will be explained in greater detail below with reference to FIGS. 36A and 36B.

FIGS. 36A and 36B are schematic views respectively illustrating the compression-distraction tool 900 in use, i.e., in distraction and compression modes, respectively.

First, the user rotates the adjusting handle 940 to position the tower supporter 930 approximately parallel to the first and second working towers 100a, 100b. For reference, if the compression-distraction tool 900 is used for minimally invasive spinal surgery, the user of the compression-distraction tool 900 according to the fourth embodiment of the invention may be a surgeon.

Next, for distraction mode in which a distance between the first and second correction screws 10a, 10b provided on the lower ends of the first and second working towers 100a, 100b is spread, the user places the pivot 910 at a location lower than the compressing body 923 and inserts the tower supporter 930 in between the first and second working towers 100a, 100b, and then referring to FIG. 36A, rotates the driving handle 927, according to which the first and second correction screws 10a, 10b are spread from each other.

Meanwhile, for the compression in which the distance between the first and second correction screws 10a, 10b provided on the lower ends of the first and second working towers 100a, 100b is reduced, the user places the pivot 910 at a location higher than the compressing body 923 and inserts the tower supporter 930 in between the first and second working towers 100a, 100b, and then referring to FIG. 36B, rotates the driving handle 927, according to which the first and second correction screws 10a, 10b are approached to each other.

The foregoing embodiments and advantages are merely exemplary and are not to be construed as limiting the present invention. The present teaching can be readily applied to other types of apparatuses. Also, the description of the exemplary embodiments of the present inventive concept is intended to be illustrative, and not to limit the scope of the claims, and many alternatives, modifications, and variations will be apparent to those skilled in the art.

What is claimed is:

1. A working tower for gripping a correction screw during minimally invasive surgery and playing a role of a working surface for other external devices, the working tower comprising:
an exterior pipe;
an interior pipe slidably provided within the exterior pipe;
a driving sleeve rotatably connected to the exterior pipe and screw-coupled to the interior pipe to move the interior pipe upward and downward along a lengthwise direction thereof during rotation; and
a screw holder provided on a lower end of the interior pipe, and is elastically split apart to receive a head portion of the correction screw when withdrawn from the exterior pipe, and contracted to securely hold the head portion when inserted into the exterior pipe.

2. The working tower of claim 1, wherein a lower half of the exterior pipe is branched into a first and a second exterior pipe branch portions, a lower half of the interior pipe is branched into a first and a second interior pipe branch portions, and a first rod guiding groove is formed between the first and second exterior pipe branch portions and a second rod guiding groove is formed between the first and second interior pipe branch portions at a location corresponding to the first rod guiding groove, whereby a rod is guided along the first and second rod guiding grooves.

3. The working tower of claim 2, wherein the screw holder comprises a first and a second holding arms each provided on ends of the first and second interior pipe branch portions and each comprises a coupling portion formed thereon to couple with the head portion.

4. The working tower of claim 3, wherein, when the first and second holding arms are elastically spread apart upon being withdrawn from the exterior pipe, a spread distance is greater than an external diameter of the head portion.

5. The working tower of claim 3, wherein the interior and exterior pipes are slid by a pipe guiding portion in an upward and downward direction without being rotated in a leftward or rightward direction in relation to each other, and the pipe guiding portion is a interior pipe guiding groove which is formed on an inner surface of each of the first and second exterior pipe branch portions to guide the first and second interior pipe branch portions.

6. The working tower of claim 5, comprising a holding arm seating recess formed on an inner surface of an end of each of the first and second exterior pipe branch portions to receive the first and second holding arms therein, wherein the holding arm seating hole is connected to the interior pipe guiding groove.

7. The working tower of claim 1, comprising a male screw formed on an upper inner circumference of the driving sleeve, a female screw for sleeve coupling formed on an outer circumference of the interior pipe to correspond to the male screw, and a female screw for external device coupling formed on the outer circumference of the interior pipe at a location spaced away from the female screw for sleeve coupling for screw-coupling with a first external device.

8. The working tower of claim 7, wherein the female screw for external device is formed on the interior pipe at a location higher than the female screw for sleeve coupling.

9. The working tower of claim 8, wherein, when the male screw is positioned between the female screw for external device coupling and the female screw for sleeve coupling, the screw holder is inserted to or withdrawn from the exterior pipe by pulling or pushing the interior pipe, and when the female screw for sleeve coupling is locked at a lower end of the driving sleeve by the pushing on the interior pipe, the screw holder is completely withdrawn from the exterior pipe, and when the female screw is coupled with the female screw for sleeve coupling after the pulling of the interior pipe, the screw holder is completely inserted into the exterior pipe.

10. A rod inserter for gripping a rod and seating the rod on a rod accommodating part of at least two correction screws during a minimally invasive surgery, the rod inserter comprising:
a fixed bar;
a moving bar slidably provided on the fixed bar;
an adjusting knob rotatably connected to the fixed bar and screw-coupled to the moving bar to adjust a moving distance of the moving bar by rotation thereof; and
a rod holder provided between one end of the fixed bar and one end of the moving bar to grip the rod in accordance with a movement of the moving bar.

11. The rod inserter of claim 10, wherein the rod holder comprise a hook provided on one end of the moving bar; and a rod supporter provided on one end of the fixed bar, wherein the rod supporter grips a center of the rod in cooperation with the hook when the hook is moved toward the adjusting knob.

12. The rod inserter of claim 11, wherein the rod holder comprises a first holding recess formed in an end of the rod supporter; and a second holding recess formed in an end of the hook to grip an end of the rod in cooperation with the first holding recess.

13. The rod inserter of claim 12, wherein the fixed bar includes a fixed handle formed on an external surface, the moving bar and the fixed bar have bent shapes due to presence of a first and a second bent portions, the fixed handle and the rod holder are positioned on different lines from each other due to the first and second bent portions, and the different lines are parallel to each other.

14. The rod inserter of claim 13, wherein the moving bar and the fixed bar are slidably coupled with each other by a rail structure between the rod holder and the first bent portion, or the moving bar is slidably inserted into the fixed bar between the second bent portion and the adjusting knob, or the moving bar and the fixed bar are located at a distance from each other between the first bent portion and the second bent portion.

15. The rod inserter of claim 10, wherein the moving bar is connected to the fixed bar through a first movement restricting unit to restrict a moving distance of the moving bar, and
the first movement restricting unit comprises,
a stop pin formed across the fixed bar, and
a movement restricting groove formed in the moving bar, in which the stop pin is inserted, and which has one and the other ends locked with the stop pin depending on the movement of the moving bar.

16. The rod inserter of claim 10, comprising a hollow spacing portion for movement formed between the adjusting knob and the other end of the moving bar so that the moving bar is advanced sufficiently toward the adjusting knob.

17. The rod inserter of claim 10, wherein the rod holder comprises a knuckle rotatably connected at one end to one end of the moving bar; a rod holder bracket in bent shape which is rotatably connected at one end to the other end of the knuckle, and rotatably connected at the other end to one end of the fixed bar; and a clip fixed to the rod holder bracket to grip an end of the rod, wherein when the moving bar moves towards the knuckle, the rod holder bracket and the clip are rotated with respect to one end of the fixed bar so that the rod gripped by the clip is rotated together.

18. The rod inserter of claim 17, wherein the moving bar comprises a cover which is slidable on an outer surface there of in a lengthwise direction, the cover comprising,
a cover body which covers the fixed bar, and
a clip locking portion provided on one end of the cover body to cover the clip so as to prevent spreading of the clip when the cover is moved in a direction toward the clip.

19. The rod inserter of claim 18, wherein the cover is connected to the fixed bar through a first multi-step adjusting unit, and
the first multi-step adjusting unit comprises a first ball elastically supported on the fixed bar; and a plurality of first locking holes formed in the cover body so that the first ball is locked therein in multiple steps.

20. The rod inserter of claim 19, wherein the cover is connected to the fixed bar through a second movement restricting unit to restrict a moving distance of the cover, and
the second multi-step adjusting unit comprises at least one long hole formed along a lengthwise direction of the cover, and at least one stop protrusion provided on the fixed bar to be moved in the at least one long hole.

21. The rod inserter of claim 18, wherein the clip locking portion comprises a rod passing hole to allow the rod to rotate efficiently without being interfered with the clip locking portion when the clip is locked by the clip locking portion and the moving bar is moved toward the clip.

22. A rod reducer for pressing down a rod seated in a rod accommodating part of a correction screw and concurrently coupling a set screw to the rod accommodating part during a minimally invasive surgery, the rod reducer comprising:
an exterior member in a tubular form comprising a set screw mounting part formed on a lower end thereof;
an interior member slidably provided inside the exterior member, the interior member comprising a lower withdrawing part formed on a lower end thereof which is in a state of being withdrawn from the exterior member into a contact with the rod; and
a compressing sleeve for coupling for descending the interior member while being coupled to a third external device gripping the correction screw so that the rod is compressed by the lower withdrawing part of the interior member; and
a rotation handle for rotating the exterior member so that the set screw is screw-coupled to the rod accommodating part of the correction screw.

23. The rod reducer of claim 22, wherein the third external device is in tubular form, and the exterior member is inserted into the third external device, and an inner circumference of the coupling sleeve is screw-coupled with an outer circumference of the third external device.

24. The rod reducer of claim 23, wherein the coupling sleeve is connected to the exterior member and the interior member by a connect unit, and the connect unit comprises,
a piercing hole formed through the interior member,
a connect pin inserted into the piercing hole,
a sliding recess formed in the exterior member to receive therein both ends of the connect pin, the sliding recess being elongated circumferentially in the inner circumference of the coupling sleeve,
a mount recess formed along a circumferential direction on an inner circumference of the coupling sleeve, and
a rotation ring rotatably mounted on the mount recess, the rotation ring in which a first and a second insertion holes are formed to receive therein both ends of the connect pin.

25. The rod reducer of claim 24, wherein the length of the sliding recess is greater than the length of the lower withdrawing part of the interior member with reference to a lengthwise direction of the exterior member.

26. The rod reducer of claim 24, wherein the interior member is elastically supported on the exterior member so that the lower withdrawing part of the interior member is maintained in a withdrawn state from the exterior member as long as the set screw is not coupled to the rod accommodating part of the correction screw.

27. The rod reducer of claim 26, wherein the interior and exterior members are elastically supported on each other via the coupling sleeve and the rotation handle, respectively, and an elastic member is provided between the coupling sleeve and the rotation handle for the elastic support.

28. A compression-distraction tool for spreading a first and a second correction screws on lower ends of a first and a second working towers from each other in a distraction mode, or approaching the first and second correction screws to each other in a compression mode in a minimally invasive surgery, the compression-distraction tool comprising:
a pivot positioned between the first and second working towers;
a tower compressing unit provided on one end of the pivot to compress the first and second working towers so that the first and second working towers are tilt with reference to the pivot; and
a tower supporter rotatably provided on the other end of the pivot to prevent movement of the first and second working towers in a lengthwise direction of the pivot.

29. The compression-distraction tool of claim 28, further comprising an adjusting handle fixed on the tower supporter in a rotatable manner about the pivot to rotate the tower supporter.

30. The compression-distraction tool of claim 29, wherein the adjusting handle is connected to the pivot through a second multi-step adjusting unit, and
the multi-step adjusting unit comprises a second ball elastically supported on the pivot; and a plurality of second locking holes formed in an outer circumference of the adjusting handle so that the second ball is locked therein in multiple steps.

31. The compression-distraction tool of claim 28, wherein the tower compressing unit comprises:
a connect bracket vertically provided on one end of the pivot;
a fixation protrusion provided on the connect bracket to be fit into the external device insertion hole of the first working tower;
a compressing body provided on the connect bracket at a predetermined distance from the pivot;
a screw rod rotatably provided on the compressing body;
a compressing nut screw-coupled with the screw rod;
a compressing protrusion provided on the compressing nut and placed on one side of the second working tower to pull the second working tower; and
a driving handle rotatably provided on an exterior side of the compressing body to rotate the screw rod so that compressing nut is moved

32. The compression-distraction tool of claim 31, wherein the compressing body is positioned vertically with respect to the pivot and the connect bracket, respectively, and elongated in a direction to run across the first and second working towers, and both ends of the compressing body is bent to a direction that is different from a direction facing the first and second working towers.

33. The compression-distraction tool of claim 28, wherein the tower compressing unit further comprises a first and a second sub-shafts rotatably provided on both outer ends of the compressing body, and the driving handle is coupled to either one of the first and second sub-shafts to rotate the screw rod.

34. The compression-distraction tool of claim 28, for distraction mode in which the first and second correction screws provided on the lower ends of the first and second working towers are spread from each other, the pivot is positioned lower than the compressing body before compressing, or for compression mode in which the first and second correction screws provided on the lower ends of the first and second working towers are approached to each other, the pivot is positioned higher than the compressing body before compressing.
